(19) United States
(12) Patent Application Publication
(10) Pub. No.: US 2012/0101331 A1
(43) Pub. Date: Apr. 26, 2012

(54) APPARATUS FOR DELIVERY OF AUTONOMOUS IN-VIVO CAPSULES

(76) Inventors: Zvika Gilad, Haifa (IL); Dan Rottenberg, Haifa (IL); Boaz Manash, Givat Ada (IL)

(21) Appl. No.: 13/322,845

(22) PCT Filed: May 27, 2010
(86) PCT No.: PCT/IL2010/000424

§ 371 Date: Nov. 28, 2011

Related U.S. Application Data
(60) Provisional application No. 61/181,838, filed on May 28, 2009.

(51) Int. Cl. A61B 1/00

(52) U.S. Cl. 600/114

(57) ABSTRACT

A guide for an endoscope capsule includes a hollow sleeve. The distal end of the sleeve has attached an invertible member for fitting the capsule within. The invertible member may be inverted via hydraulic or pneumatic pressure to expel the capsule from the guide and into a desired location within a patient's body lumen. The guide may be attached to an actuator which contains a fluid and an actuating member. The actuating member pressurizes the fluid distally through the hollow sleeve thereby inverting the invertible member and expelling the capsule into the body lumen. The guide may be used with an endoscope or may be a stand-alone device.

### FIELD OF THE INVENTION

The present invention relates to an apparatus for the delivery into a body lumen of autonomous in-vivo capsules that are to be used in internal imaging of the body lumen.

### BACKGROUND OF THE INVENTION

Endoscopic and other insertion devices for delivering into a body lumen, such as a gastro-intestinal tract, an autonomous capsule, such as an imaging capsule, are known in the art. Some examples of such devices are, for example, described in: U.S. Pat. No. 6,632,171 (Iddan), U.S. Pat. No. 6,884,213 (Raz), U.S. Pat. No. 5,653,677 (Okada), U.S. Pat. No. 5,681,279 (Roper), U.S. Pat. No. 5,630,782 (Adair), U.S. Pat. No. 5,489,256 (Adair), U.S. Pat. No. 6,984,205 (Gazdzinski) and U.S. Pat. No. 7,001,329 (Kobayashi); U.S. Patent Application Publication No. 2005/0267361 (Younker), U.S. Patent Application Publication No. 2007/0055097 (Kura; Yasuhito), and U.S. Patent Application Publication No. 2005/0183733 (Kawano, Hironao); and International Patent Application Publication No. WO 2005/032352 (Yokoi).

In the aforementioned examples wherein the capsule is releasable, the autonomous capsule is fixedly attached to the distal end of an endoscopic or other insertion device by mechanical, magnetic or other means, and is guided through the body cavity by a pushing force exerted on the proximal, i.e., external, end of the insertion device. The insertion device may be flexible, allowing it and the attached capsule to approximately conform to the natural shape of the interior surface of the body cavity as it is moved therethrough. Once the distal end of the insertion device reaches the desired location within the body lumen, the autonomous capsule is released by release of the mechanical, magnetic or other means by which it was fixedly attached to the insertion device.

This approach presents problems. Some solutions, such as Okada, Yokoi and Kobayashi, require redesign of the imaging capsule in order to fit the release mechanism. This is a major disadvantage to a capsule delivery device that should be designed to work with various imaging capsules that are available in the market, such as the PillCam® capsule endoscopes of Given Imaging Ltd.

In addition, in hydraulic or pneumatic release mechanisms, such as in Raz or Younker, the capsule must be held in the delivery device tightly enough so that the capsule is not prematurely released before reaching the target point. Accordingly, the pressure that is needed to release the capsule must be quite strong, and as a result the capsule is forcefully propelled from the delivery device. This forceful release of the capsule is undesirable, as it could damage the capsule or, worse, cause damage to the patient.

Another problem is the inability of the operator of the insertion device, prior to release of an autonomous imaging capsule from the endoscopic device, either before or after the insertion device has reached the desired location within the body lumen, to manipulate the angle/direction of view of the imaging capsule independently from the orientation of the insertion device as a whole. For example, the operator may desire to view the body lumen along the way to the desired location within the body lumen and prior to release of the autonomous capsule or may desire to view a portion of the body lumen proximate to the location at which the autonomous capsule is to be released. Thus, the operator of the insertion device may desire to utilize the imaging capability of the capsule to be released prior to its release, and the operator should be able to manipulate the angle/direction of view of the imaging capsule independently from the orientation of the insertion device as a whole. However, in all of the aforementioned examples, in order to change the angle/direction of view of the capsule, the insertion device as a whole must be pushed, pulled and/or rotated. Such manipulations of the insertion device, especially at bends in the gastro-intestinal tract, may cause additional medical risk to the body.

U.S. Patent Application Publication No. 2005/0085697 to Yokoi et al. describes means to control the angle/direction of view of the capsule independently of the orientation of the insertion device as a whole. In Yokoi, the imaging capsule is attached to the insertion device by two strings extending through a housing of the insertion device body. At the capsule, each of the two strings is attached off-center from one of the axes of symmetry of the capsule. Thus, when a string is tugged, a torsional force pivots the capsule about an axis orthogonal to that axis of symmetry, with one string pivoting the capsule in one direction, and the other string pivoting the capsule in another direction. One problem with this device is that it requires a special purpose capsule, namely, one with holes for the strings, and cannot be used with any other capsule. Another problem with this device is that the capsule can be moved only with one degree of freedom relative to the insertion device, as the capsule moves (pivots) only in the plane in which both of the strings are attached to the capsule. The only way for the capsule to rotate away from this plane is for the whole insertion device to be rotated. Another problem with the device of Yokoi is that it is difficult for a doctor or other operator of the apparatus to dexterously manipulate the two strings and the insertion device concurrently.

There is, therefore, a need in the art for an insertion apparatus for delivery of an autonomous imaging capsule that enables the capsule, prior to release thereof from the insertion device, to be capable of being independently oriented in all directions relative to the insertion device, while also providing ergonomic means of control.

There is also a need in the art for an insertion apparatus for delivery of an autonomous imaging capsule, such as one with an optical head at each end, that has a reduced likelihood of damaging the relatively vulnerable window of the imaging capsule by the release mechanism.

### SUMMARY OF THE INVENTION

Accordingly, there is now provided with this invention an improved insertion apparatus for delivery of an autonomous capsule that effectively overcomes the aforementioned difficulties and longstanding problems in the art.

In one embodiment of the invention, a guide for an endoscope capsule may comprise a hollow sleeve having a proximal end and a distal end. The guide may further comprise an attachment element for mounting the capsule. The attachment element may be attached to the distal end of the sleeve, and the attachment element may have a cavity. The guide may further comprise an invertible member for fitting the capsule within. In some embodiments, the invertible member may be positioned within the cavity and attached to the attachment element. The invertible member may be inverted via hydraulic or pneumatic pressure to expel the capsule from the attachment element.

In some embodiments of the invention, the guide may further comprise an actuator. The actuator may comprise a cavity containing a fluid and an actuating member to pressurize the fluid in the cavity, thereby inverting the invertible member.

In some embodiments of the invention, the fluid within the actuator may be selected from a group consisting of: water, saline solution and air.

In some embodiments of the invention, the sleeve may comprise a mating element for securing said mounting element onto the sleeve. In some embodiments, the mating element may be attached to the sleeve by attachment means selected from a group consisting of: a luer lock, a clip, a snap, a detent mechanism, a screw and a magnet.

In some embodiments of the invention, the guide may be contained within an endoscope. In other embodiments, the guide may be a stand-alone device that need not be used with an endoscope.

### BRIEF DESCRIPTION OF THE DRAWINGS

The above and other objects and advantages of the invention will be understood and appreciated more fully upon consideration of the following detailed description, taken in conjunction with the accompanying drawings, in which the reference characters refer to like parts throughout and in which:

FIG. 1 shows a back and side perspective view of a distal end of a guide apparatus for moving an attached capsule through the gastro-intestinal tract;

FIGS. 2A and 2B show a full side view and a cross-sectional side view, respectively, of the guide apparatus and attached capsule of FIG. 1;

FIGS. 3A and 3B show a full side view and a cross-sectional side view, respectively, of the guide apparatus and attached capsule of FIG. 1 in a straightened configuration;

FIG. 4 shows the degrees of movement of the guide apparatus of FIG. 1;

FIGS. 5A and 5B show back and side perspective views of the guide apparatus of FIG. 1 with and without an attachment member, respectively;

FIG. 6 shows a side view of the guide apparatus and attached capsule of FIG. 1 when used with an endoscope;

FIGS. 7A and 7B show a full view and a cross-sectional view, respectively, of the guide apparatus of FIG. 1 having a mechanism for releasing the capsule according to a first embodiment;

FIGS. 8A and 8B show the guide apparatus of FIG. 1 having a mechanism for releasing the capsule from the attachment member according to a second embodiment;

FIGS. 9A and 9B show cross-sectional side views of the attachment member for holding and releasing the capsule, respectively;

FIGS. 10A and 10B show back and side perspective views of two distinct embodiments of the attachment member;

FIGS. 11A and 11B show a back and side perspective view and a cross-sectional view, respectively, of a third embodiment of the attachment member;

FIGS. 12A and 12B show cross-sectional views of a fourth embodiment of the attachment member;

FIGS. 13A and 13B show perspective views of an invertible member for use in the fourth embodiment of the attachment member shown in of FIGS. 12A and 12B;

FIGS. 14A and 14B show perspective views of the attachment member with an invertible member disposed within the cavity thereof before and after release of the capsule therefrom;

FIGS. 15A and 15B show cross-sectional views of the attachment member with an invertible member disposed within the cavity thereof before and after release of the capsule therefrom;

FIGS. 16A, 16B, and 16C show front views of the guide apparatus using mechanism for releasing the capsule from the attachment member according to a fifth embodiment; and

FIGS. 17A, 17B, and 17C show side views of the attachment member of FIGS. 16A, 16B, and 16C, respectively, during release of the capsule.

FIGS. 18A and 18B show back and side and front and side perspective views of a guide apparatus and attached capsule in accordance with another embodiment of the invention;

FIGS. 18C and 18D show side perspective views of the guide apparatus and attached capsule of FIGS. 18A and 18B, respectively;

FIGS. 19A and 19B show back and side and front and side perspective views of a guide apparatus and attached capsule in accordance with yet another embodiment of the invention;

FIGS. 19C and 19D show a full side view and a cross-sectional side view, respectively, of the guide apparatus and attached capsule of FIGS. 19A-19B;

FIGS. 20A and 20B show back and side and front and side perspective views of a guide apparatus and attached capsule in accordance with a third embodiment of the invention;

FIGS. 20C and 20D show side perspective views of the guide apparatus and attached capsule of FIGS. 20A and 20B, respectively;

FIGS. 21A and 21B show front and side and back and side perspective views of a guide apparatus and attached capsule in accordance with a fourth embodiment of the invention;

FIG. 22A shows a front view of the guide apparatus using mechanism for controlling orientation of the capsule according to an embodiment of the invention;

FIGS. 22B and 22C show a back and side perspective views of the guide apparatus and mechanism for controlling orientation of the capsule of FIG. 22A;

FIG. 23A shows a front view of the guide apparatus using mechanism for controlling orientation of the capsule according to another embodiment of the invention; and

FIGS. 23B and 23C show a back and side perspective views of the guide apparatus and mechanism for controlling orientation of the capsule of FIG. 23A.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

The device of the present invention may be used with an autonomous imaging system or device such as that described in U.S. Pat. No. 5,604,531 entitled “In Vivo Video Camera System,” which is incorporated herein by reference. Another example of an imaging system and device with which the device of the present invention may be used is described in U.S. Pat. No. 7,009,634 entitled “Device for In Vivo Imaging,” which is incorporated herein by reference. A further example of an imaging system and device with which the device of the present invention may be used is described in U.S. Patent Application Publication No. 2007/0118018 entitled “In-Vivo Imaging Device and Optical System Thereof,” and U.S. Patent Application Publication No. 2007/0118012 entitled “Method of Assembling an In-Vivo Device”, which are incorporated herein by reference. For example, a swallowable imaging capsule having an imager at one or both ends, such as that described in one of these publications, or any of the PillCam® capsule endoscopes of Given Imaging Ltd. may be used in the present invention.

The imaging capsule may be an autonomous imaging capsule, as discussed above, that includes one or more light sources, a viewing window through which the light sources illuminate inner surfaces of the digestive system, an imaging system which detects the images, an optical system which focuses the images onto the imaging system, a transmitter which transmits the image data from the imaging system, and a power source, such as a battery, which provides power to the entirety of electrical elements of the capsule. The capsule may additionally or alternatively include sensor elements for measuring pH, temperature, pressure, etc., as is known in the prior art.

Typically, such a capsule is swallowed by the patient and passes through the patient's gastrointestinal tract, while transmitting signals relating to data, e.g., image data, concerning the gastrointestinal (GI) tract sensed by the capsule. There are times, however, when it is desired to assist patients having difficulty swallowing the capsule. In addition, it may also be desired to deposit an imaging capsule at a specific location within the GI tract and to use the imaging capsule as a manipulable endoscope prior to release of the imaging capsule into the gastrointestinal tract. For example, using a guide apparatus to guide the capsule to the target location within the GI tract may reduce the time required for the capsule to reach the target location and may also enable use of the capsule for more detailed and sustained imaging than would be accomplished by the autonomous progression of the capsule at the target location. For this purpose, the capsule is temporarily detachably attached to a guide apparatus, typically in the form of an endoscopic tube member that is inserted into the patient's body lumen.

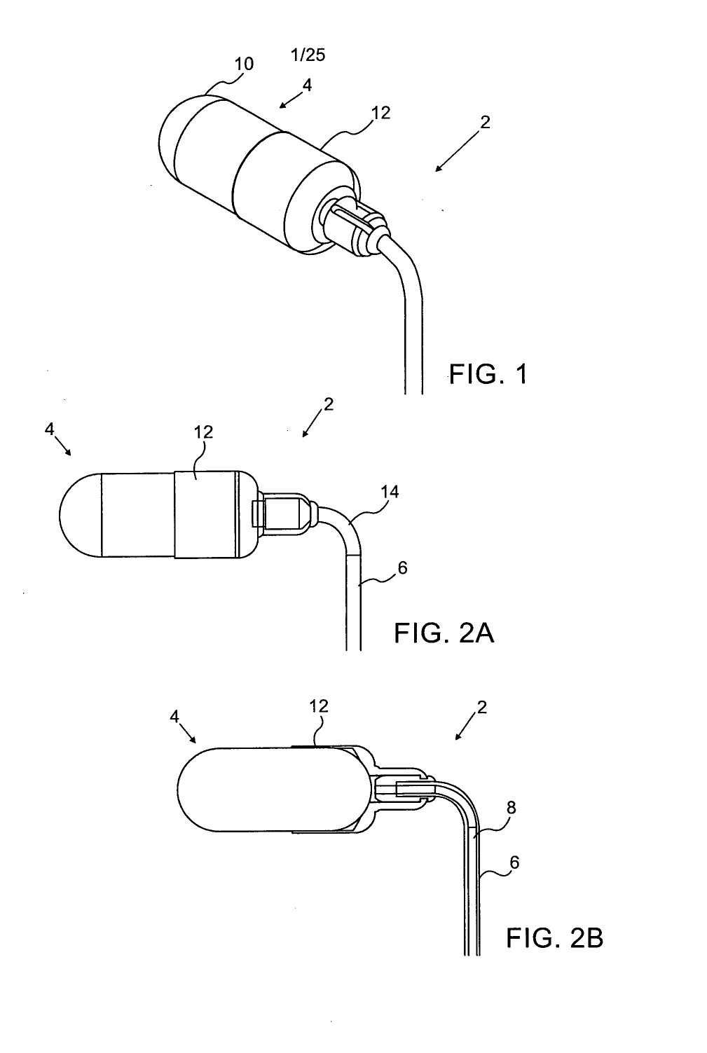

Reference is made to FIG. 1, showing a back and side perspective view of a distal end of a guide apparatus 2 for moving a swallowable, autonomous capsule 4 through the GI tract. Guide apparatus 2 may include an attachment member 12 for mounting capsule 4 onto guide apparatus 2, in a preferred embodiment without any alteration to the capsule 4. Attachment member 12 may be any attachment member that fixedly attaches the autonomous capsule 4 to guide apparatus 2 by mechanical force (friction), vacuum force, or other means, as is known in the art.

In the embodiment shown in FIG. 1, attachment member 12 is a holding cup with a mechanical release mechanism, as discussed hereinbelow. Capsule 4 is releasably placed into attachment member 12 and is held sufficiently securely therein such that capsule 4 is not released from attachment member 12 during manipulation of guide apparatus 2 through the GI tract.

Guide apparatus 2 may be used to deliver capsule 4 to a target location within the GI tract. Once the target location is reached, capsule 4 is released from attachment member 12 of guide apparatus 2 and travels autonomously throughout the remainder of the GI tract until it is excreted. Capsule 4 should preferably be removable with only a mild force so that the physician need not exert significant force to release capsule 4 when outside the patient's body. Capsule 4 should be released from guide apparatus 2 smoothly when in vivo, as forcible ejection of capsule 4 off guide apparatus 2 carries risk of inconvenience to the patient and damage to sensitive body tissue.

Reference is made to FIGS. 2A and 2B, showing a side view and a cross-sectional view, respectively, of guide apparatus 2 of FIG. 1.

Guide apparatus 2 may include a hollow annular sleeve 6 and a shaft 8 traversing the annulus of hollow sleeve 6 (shaft 8 is visible only in the cross-sectional view of FIG. 2B). Shaft 8 is adapted to move longitudinally (i.e., slide) as well as to be rotated (i.e., twisted) relative to and within sleeve 6. Shaft 8 may be manipulated remotely from sleeve 6, i.e., from outside the patient's body when shaft 8 is inserted within the body, to turn it within sleeve 6, to push it towards the distal end of sleeve 6 or to retract it towards the proximal end of sleeve 6. Such rotation, pushing and pulling of shaft 8 relative to sleeve 6 causes a change in the angle/direction of view of capsule 4, as discussed in greater detail below. Such manipulation can be performed by using a handle, knob or other similar device at the proximal end of shaft 8, such as are well known in the art.

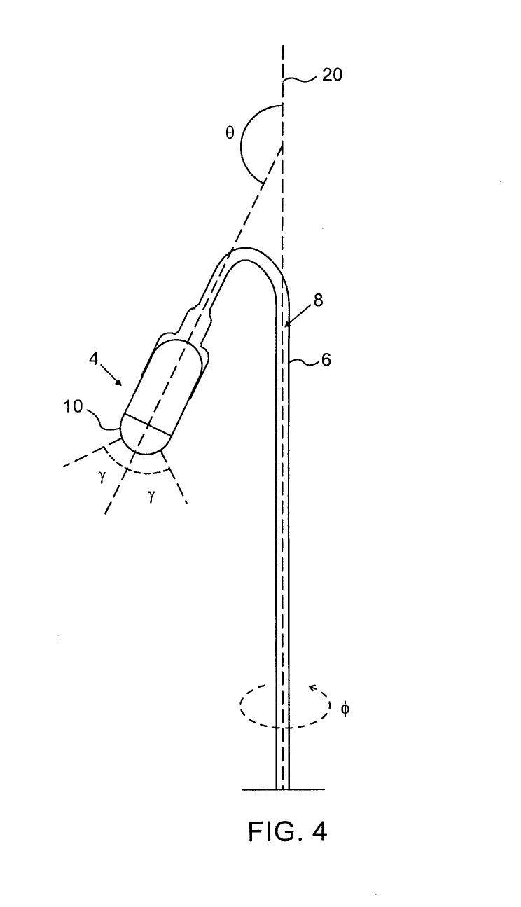

In its relaxed state (when not being acted upon by external forces), the distal end of sleeve 6 may have a bend 14 at an angle relative to the longitudinal axis of sleeve 6. In certain embodiments, sleeve 6 may be bent at an angle approximately equal to 90°, as shown in FIGS. 2A and 2B. In other embodiments, sleeve 6 may be bent at an angle of less than 90° or of greater than 90°, for example anywhere up to approximately 170° and preferably, approximately 135°, as shown in FIG. 4.

Despite its naturally bent shape, sleeve 6 has sufficient flexibility to allow it to be deformed, preferably by straightening bend 14 or by bending bend 14 further, when acted upon by external forces, while having a sufficient spring constant to return to its preformed, bent shape when the external forces are removed.

In one embodiment of the present invention, shaft 8 is more rigid than sleeve 6 within which shaft 8 is enclosed, such that the rigidity of shaft 8 dominates and determines the shape of sleeve 6. In one embodiment, shaft 8 may be straight. Thus, when the straight shaft 8 moves through bend 14 of sleeve 6, shaft 8 provides a straightening force on sleeve 6 that causes sleeve 6 to conform to the straight shape of shaft 8.

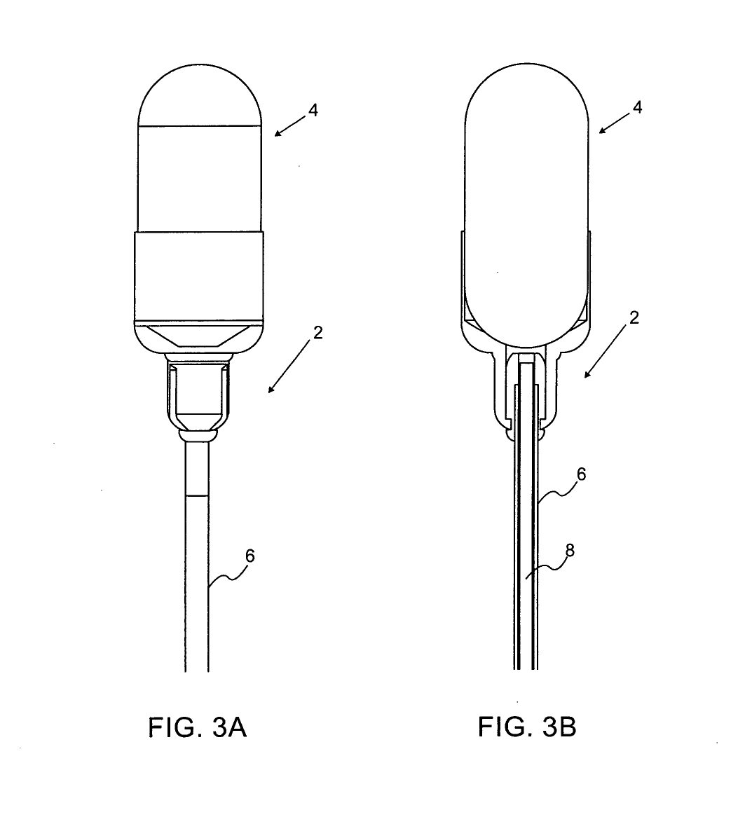

Reference is made to FIGS. 3A and 3B, showing an outside view and a cross-sectional view of guide apparatus 2 of FIG. 1, respectively, being straightened by the extension of shaft 8 within and relative to sleeve 6. When shaft 8 is extended towards the distal end of the hollow sleeve 6, as in FIGS. 3A and 3B, the shape of sleeve 6 conforms to the shape of shaft 8. When shaft 8 is retracted back towards the proximal end of sleeve 6, as in FIGS. 2A and 2B, sleeve 6 is relaxed to its natural bent shape.

The inherent structure of the body lumen provides an additional external force to deform the shape of sleeve 6. Preferably the body lumen is more rigid than both sleeve 6 and shaft 8 combined, such that both sleeve 6 and shaft 8 conform to the shape of the GI tract when inserted therethrough.

Referring again to FIGS. 2A and 2B, when shaft 8 is manipulated such that it is retracted back towards the proximal end of sleeve 6, there is no substantial force at bend 14 to prevent sleeve 6 from returning to its natural bent shape, such that sleeve 6 will return to its preformed shape when the forces of shaft 8 are removed. In addition, even a portion of sleeve 6 will return to its preformed shape when the forces of shaft 8 are removed from that portion. Thus, while the portion of sleeve 6 traversed by shaft 8 still conforms to the shape of shaft 8, the portion of sleeve 6 not traversed by shaft 8, i.e., from which shaft 8 has been retracted, experiences no straightening force from shaft 8 and, to the extent not also restrained by the body lumen, returns to its preformed shape, as discussed above.

Thus, the angle of bend 14 of guide apparatus 2 may be manipulated by altering the length of shaft 8 extending into the region defining bend 14 of sleeve 6. Because bend 14 is a gradual curve and extends along a length of sleeve 6, the length of a portion of shaft 8 that extends into bend 14 forces the same length of sleeve 6 along bend 14 to straighten. As shaft 8 is extended farther into bend 14 of sleeve 6 to straighten more and more of sleeve 6 along bend 14, the result is a decreased angle of bend 14 relative to the longitudinal axis of sleeve 6. At an extreme, shaft 8 is fully extended, and the angle of bend 14 is zero. Conversely, as shaft 8 retracts farther from bend 14 of sleeve 6 to straighten less and less of sleeve 6 along bend 14, the result is an increased angle of bend 14 relative to the longitudinal axis of sleeve 6. At an extreme, shaft 8 is fully retracted and the angle of bend 14 is the angle of the natural bend of sleeve 6.

Since attachment member 12 attaches to sleeve 6 at the tip of sleeve 6, i.e., distally along sleeve 6 relative to bend 14, attachment member 12, and therefore also capsule 4 attached thereto, are oriented in alignment with the distal end of bend 14. The angle of bend 14 may, therefore, be manipulated to alter the angle/direction of view of capsule 4.

Reference is made to FIG. 4, which shows the degrees of movement of guide apparatus 2 of FIG. 1. The angle/direction of view of capsule 4 when attached to guide apparatus 2 is affected by several parameters, such as, for example, the natural angle/direction of view of the imaging capsule 4 itself, the movement of guide apparatus 2 as a whole (i.e., pushing and pulling through the GI tract), and the movement of capsule 4 relative to guide apparatus 2.

The movement of capsule 4 relative to guide apparatus 2 may be controlled by two different manipulations: by moving shaft 8 longitudinally relative to and within sleeve 6 (i.e., in a direction of a longitudinal axis 20), and by rotating guide apparatus 2 (i.e., twisting about longitudinal axis 20) within the work channel of an endoscope. As disclosed hereinabove, moving shaft 8 longitudinally relative to and within sleeve 6 alters an angle θ of bend 14. Angle θ of bend 14 may be altered to be from 0° (when shaft 8 is fully extended) to a maximum angle, e.g., 135° (when shaft 8 is fully retracted) in the plane of bend 14. The plane of bend 14 may be changed by rotating guide apparatus 2 at an angle φ about longitudinal axis 20 of guide apparatus 2. For example, by rotating guide apparatus 2 an angle φ of up to 360°, guide apparatus 2 may be rotated through every plane in a 360° view. Attachment member 12 onto which capsule 4 is mounted may therefore be moved in a total range of, for example, ≦θ≦135° in the direction of angle θ (by moving shaft 8 relative to sleeve 6) and 0≦φ≦360° in the direction of angle φ (by rotating guide apparatus 2). This range of motion of attachment member 12 is substantially the shape of a surface of a sphere, with a hole in the space occupied by sleeve 6 itself. It may be appreciated that other angles and ranges of movement may be used.

The angle/direction of view of capsule 4 itself (i.e., the range of viewing angles γ of the imaging system through viewing window 10) allows images to be taken at angles beyond where attachment member 12 can physically reach, in order to obtain a full 180° view on each side of longitudinal axis 20. Thus, the maximum angle θ that sleeve 6 is required to bend to obtain the full 180° angle of view on one side of longitudinal axis 20 may be reduced by angle γ of the angle of view of the imaging capsule 4 itself. For example, if the angle of view of the imaging capsule 4 itself is γ=45° in all directions from the center axis of view, then the angle of sleeve 6 with respect to the longitudinal axis need only bend a maximum angle θ of 135° in that plane of view in order for the total viewing angle of guide apparatus 2 to be a full 180° to one side of the longitudinal axis 20. Sleeve 6 is then simply rotated an angle φ=180° about longitudinal axis 20 in order to obtain the view of other angles around longitudinal axis 20. Thus, the total angle of view of capsule 4 mounted in attachment member 12 is 360° in all directions in three-dimensions.

It should be understood that, although longitudinal axis 20 of guide apparatus 2 is shown to be a straight line in FIG. 4, as the endoscope within whose work channel the flexible guide apparatus 2 extends traverses the body lumen, guide apparatus 2 will bend to conform to the shape of the lumen. Longitudinal axis 20 of guide apparatus 2 may, therefore, refer to an axis traversing the portion of guide apparatus 2 that is most immediately proximal to bend 14 in its natural non-deformed state, rather than to a straight line.

In designing the shape of bend 14 of sleeve 6, there are several considerations. One consideration is to maximize the potential angle/direction of view of capsule 4. This is achieved by maximizing angle θ of bend 14, as discussed in reference to FIG. 4. However, another consideration is to minimize the force that is required to extend shaft 8 into bend 14 for changing the angle θ. The force required to extend shaft 8 into bend 14 is a function of the curvature of bend 14, not the angle θ of bend 14 itself. The larger the curvature of bend 14 (i.e., the sharper the turn of angle θ), the less leverage shaft 8 has to straighten the bent portion of sleeve 6, which therefore requires more force to extend shaft 8 into bend 14.

In one embodiment, the ideal curvature of bend 14 may depend on the relative rigidity and stiffness of shaft 8 and of sleeve 6 and on the smoothness of their respective surface materials. In preferred embodiments, the radius of curvature of bend 14 of sleeve 6 should not be too small so as to prevent shaft 8 from straightening bend 14 of sleeve 6. In this regard, in certain embodiments, shaft 8 should have sufficient rigidity and stiffness to be able to overcome the natural bend 14 of sleeve 6 so as to be able to deform sleeve 6 from its natural bent state. However, conversely, the radius curvature of sleeve 6 should not be too large so as to form an excessively gradual and long bend 14 in sleeve 6, which would require moving an equally long length of shaft 8 to straighten sleeve 6.

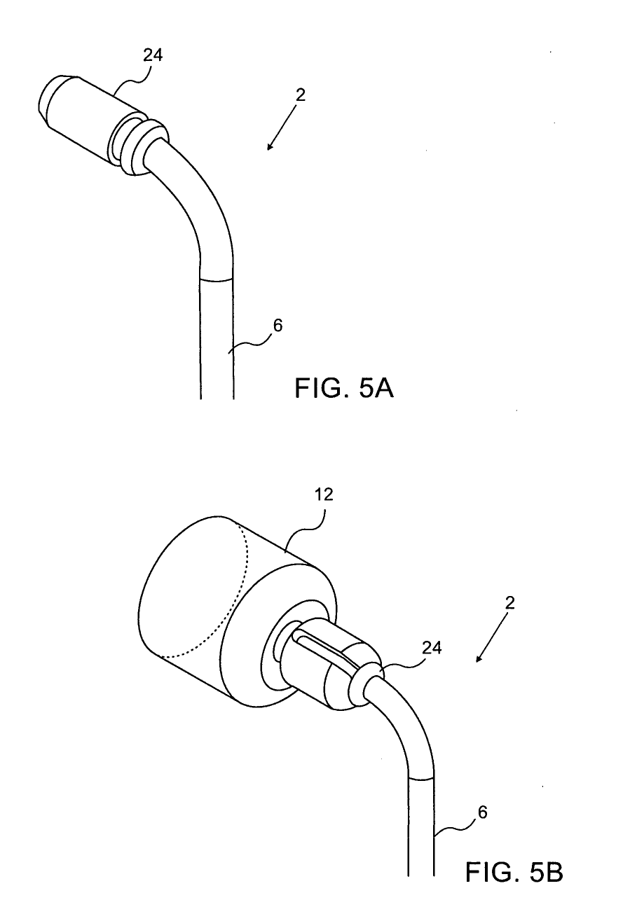

Reference is made to FIGS. 5A and 5B, showing perspective views of guide apparatus 2 of FIG. 1 with and without attachment member 12, respectively, for mounting capsule 4 onto guide apparatus 2.

FIG. 5A shows a mating element 24 on the distal tip of guide apparatus 2. Mating element 24 is adapted for securing attachment member 12 onto guide apparatus 2, as shown in FIG. 5B. Attachment member 12 and mating element 24 may attach by any attachment means, for example, a Luer lock, a clip, a snap, a detent mechanism, a screw or a magnet. In one embodiment, attachment member 12 possesses a circumferential protrusion on an inner surface thereof that mates with a circumferential groove on the outer surface of mating element 24. In another embodiment, attachment member 12 and mating element 24 may be adapted to attach and release (after capsule 4 is dispensed). Alternatively, guide apparatus 2 may be disposable and attachment member 12 may not release at all from mating element 24.

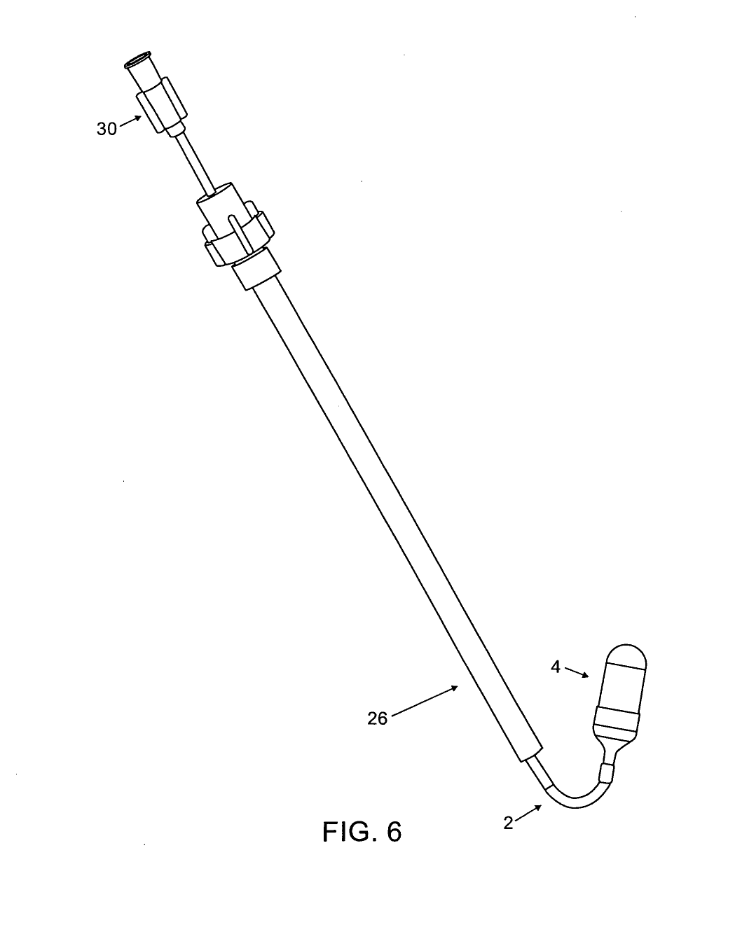

According to an embodiment of the present invention, guide apparatus 2 may be used with an endoscope. Reference is made to FIG. 6, showing guide apparatus 2 of FIG. 1 used with an endoscope 26. Guide apparatus 2 may be adapted to fit within a hollow, annular opening of endoscope 26 to allow the two devices to move together through the GI tract.

Endoscope 26 may include any hollowed endoscope that is known in the art, such as, for example, those manufactured by Olympus, Fujinon or Pentax. The opening of endoscope 26 may have a diameter of, for example, approximately 2.5-3 millimeters (mm). The outer surface of sleeve 6 of guide apparatus 2 typically has a diameter smaller than the diameter of the hollow opening of endoscope 26, for example, approximately 2 mm. Sleeve 6 has an inner opening with a diameter slightly greater than the diameter of the outer surface of shaft 8, for example, by 0.1 mm, so that they form a close-fit. Shaft 8 has a diameter of, for example, approximately less than 1 mm and, in one embodiment, preferably approximately 0.4 mm. At its widest region, attachment member 12 may have a diameter, for example, approximately 3-3.5 mm, but generally greater than the diameter of the inner opening of endoscope 26 so that attachment member 12 may be secured and held proximal to endoscope 26 without being pulled into endoscope 26.

In one embodiment, the parts described above may be assembled by first threading shaft 8 from and through the proximal opening of sleeve 6 to form guide apparatus 2 (without attachment member 12, as shown in FIG. 5A). A controller 30 may be attached to the proximal end of shaft 8 to control the movements of shaft 8 relative to sleeve 6. Initially, controller 30 may extend shaft 8 toward the distal end of sleeve 6 so as to straighten guide apparatus 2. Guide apparatus 2 may be threaded through a hollow opening of endoscope 26 until the tip of guide apparatus 2 protrudes from the distal end of endoscope 26. Attachment member 12 may be secured to sleeve 6 by way of a mating element 24 that is attached, e.g., via snap fit, at the distal tip of guide apparatus 2. Since the diameter of attachment member 12 is greater than the diameter of the inner opening of endoscope 26, endoscope 26 can be held proximal to capsule 4. Endoscope 26 may also have an endoscope imager positioned outside the body, i.e. an optical fiber. Alternatively, imaging capsule 4 may be viewed through endoscope 26, as taught in U.S. Pat. No. 6,884,213 (Raz). Typically, endoscope imager 28 is used by an administrator to determine the position of capsule 4 when delivering capsule 4 to a target location within the GI tract. Alternatively, a real-time broadcast of images taken by capsule 4 may be used instead of endoscope imager 28.

The delivery and release of capsule 4 may include several different stages of operation. In the esophagus, the administrator may grip controller 30 to keep shaft 8 distally extended to straighten bend 14 of guide apparatus 2 to prevent capsule 4 from protruding sideways as guide apparatus 2 travels through the relatively narrow opening of the esophagus. When capsule 4 reaches the relatively wide opening of the stomach, the administrator may manipulate controller 30 so as to retract shaft 8 to varying degrees to move the imaging capsule 4 to investigate the surrounding area at any angle, as described above. The administrator may also manipulate controller 30 so as rotate guide apparatus 2 in all directions to obtain a 360° view of the surrounding area. After imaging the full view of the stomach, the administrator may manipulate controller 30 so as to actuate guide apparatus 2 to release capsule 4.

In an alternative embodiment, not shown herein, the manipulation of guide apparatus 2 to deliver and change the angle/direction of view of imaging capsule 4 may be done using an inverse apparatus, i.e., wherein shaft 8 is bent and guide apparatus 2 is straight and is more rigid than shaft 8. In this embodiment, the shape of guide apparatus 2 controls the curvature of the combined guide apparatus/shaft assembly by retractably sliding forward and backward over the curved shaft 8, by manipulation by an administrator using a controller 30

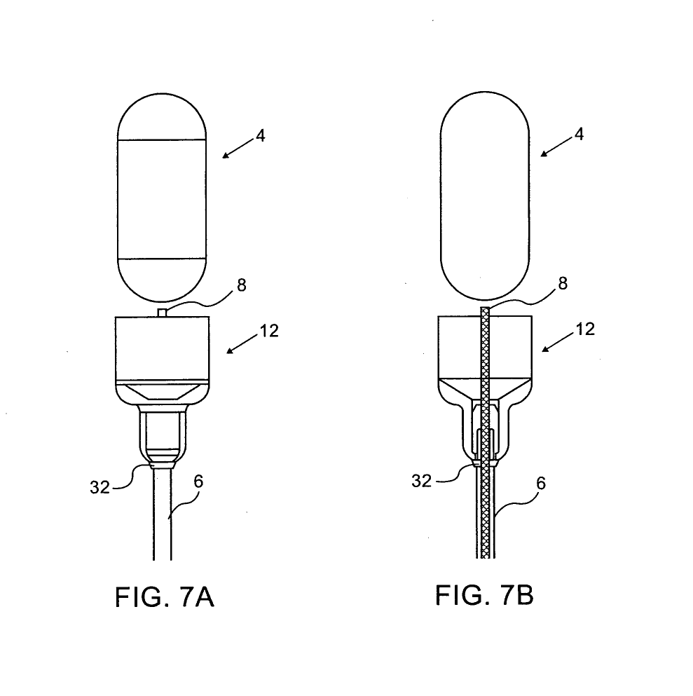

Reference is made to FIGS. 7A and 7B, showing an outside view and a cross-sectional view, respectively, of guide apparatus 2 of FIG. 1 having a first embodiment of a mechanism for releasing capsule 4 from guide apparatus 2 of FIG. 1. In this first embodiment, capsule 4 is released mechanically from guide apparatus 2. In one embodiment of the mechanical release, the hollow cavity of sleeve 6 may extend into an opening 32 at the proximal terminal of attachment member 12. To release capsule 4, an administrator may manipulate grip controller 30 to extend shaft 8 into opening 32 to abut against and force capsule 4 to dislodge from its mount. Shaft 8 should have sufficient rigidity and stiffness to be able to supply a sufficient force so as to overcome the force holding capsule 4 in attachment member 12 and to dislodge capsule 4 from attachment member 12.

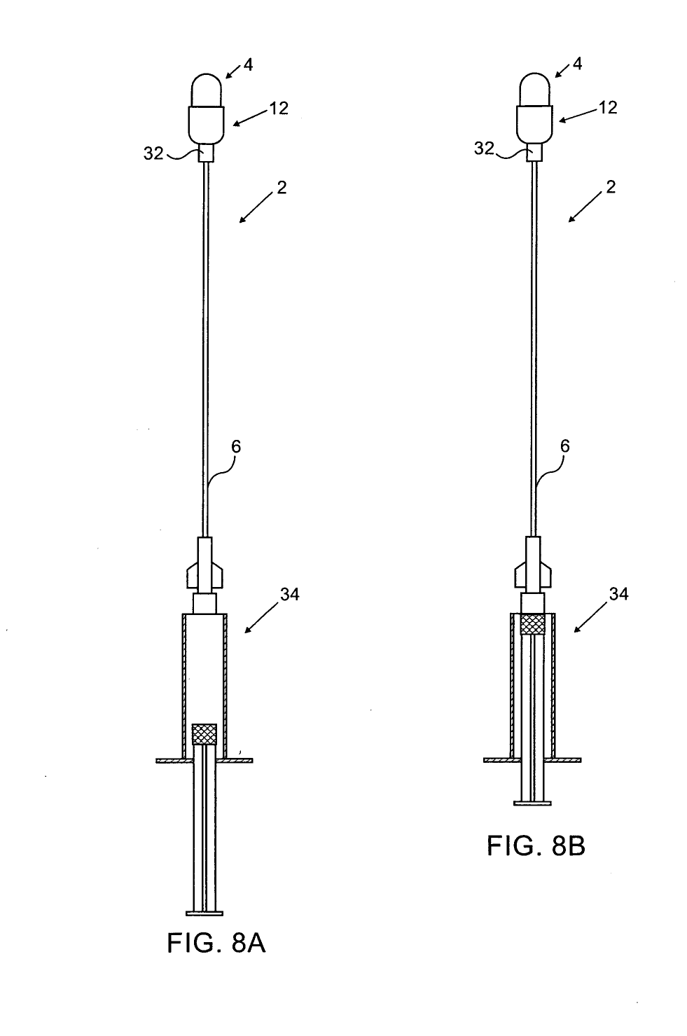

Reference is made to FIGS. 8A and 8B, showing views of guide apparatus 2 of FIG. 1 having a second embodiment of a mechanism for releasing capsule 4 from guide apparatus 2. In this second embodiment, capsule 4 is released hydraulically or pneumatically from guide apparatus 2, for example using a hydraulic or pneumatic actuator 34, as shown in FIGS. 8A and 8B. Actuator 34 may include a cavity containing a fluid, such as a liquid, e.g., water or saline solution, or gas, e.g., air, and an actuating member to pressurize the material in the cavity. When hydraulic or pneumatic actuator 34 is actuated, the pressurized fluid is forced distally through hollow sleeve 6 of guide apparatus 2. The pressurized fluid may travel through opening 32 at the proximal terminal of attachment member 12 and apply a force to capsule 4 sufficient to dislodge capsule 4 from attachment member 12. Actuator 34 may be for example a 2 cc syringe, pump, or any other device for altering pressure. Naturally, the fluid contained within the cavity of hydraulic actuator 34 should be non-toxic and suitable for release into the relevant body lumen, in embodiments wherein the fluid is perfused into the body lumen during release of capsule 4 from attachment member 12.

In FIG. 8A, the actuating member of hydraulic actuator 34 is shown as proximally retracted. In the retracted state, approximately no amount or an ineffective amount of net pressure is exerted in the cavity of hydraulic actuator 34. Thus, no force is applied to release capsule 4. Instead, capsule 4 is fit within the cavity of attachment member 12, which secures capsule 4 to guide apparatus 2, as described above, e.g., via frictional forces.

In FIG. 8B, the actuating member of hydraulic actuator 34 is shown as distally extended. In the extended state, a sufficient external force is applied to the material contained in the cavity of the hydraulic actuator 34 to force the material distally so as to dislodge capsule 4 from attachment member 12. To release the capsule 4, hydraulic actuator 34 must supply a force at least greater than the attachment force, e.g., a frictional force, between attachment member 12 and capsule 4.

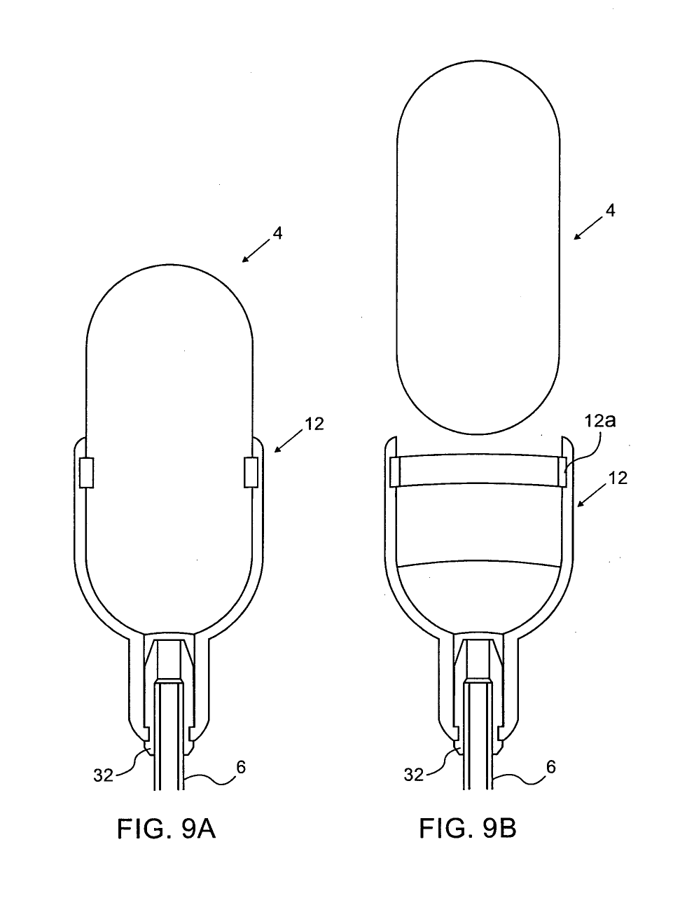

Reference is made to FIGS. 9A and 9B, showing cross-sectional views of attachment member 12. As shown in FIGS. 9A and 9B, attachment member 12 may be attached to and released from the distal end of guide apparatus 2. In FIG. 9A, capsule 4 is shown as secured to attachment member 12 via frictional forces. In FIG. 9B, capsule 4 is shown as released from attachment member 12.

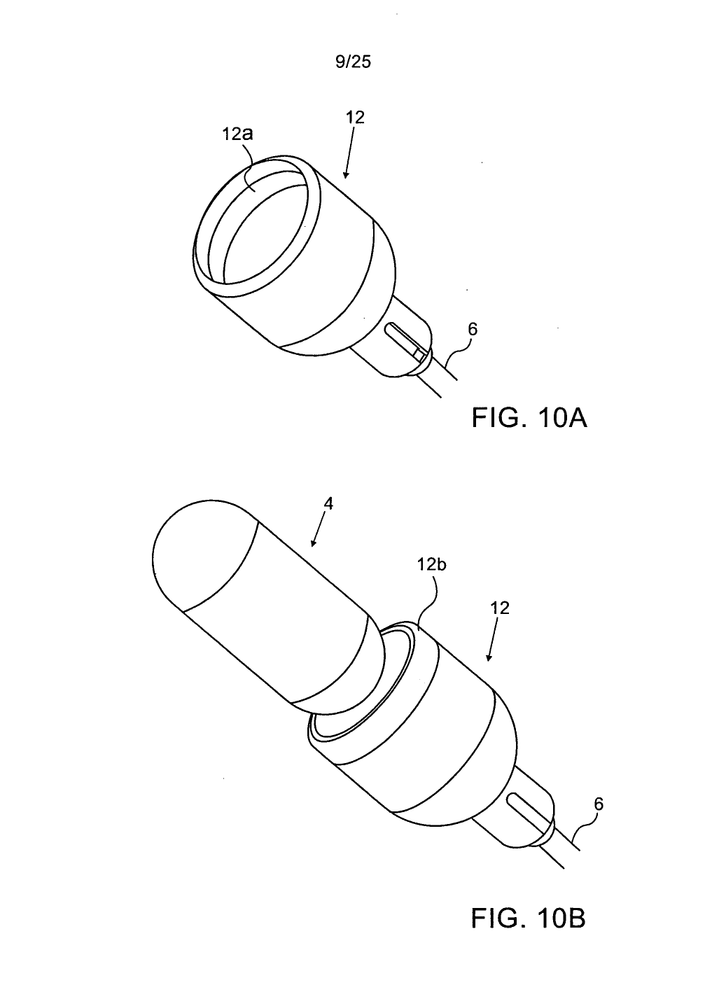

Reference is made to FIGS. 10A and 10B, showing perspective views of two distinct embodiments of attachment member 12 as shown in FIGS. 9A and 9B wherein capsule 4 is secured to attachment member 12 via frictional forces. As shown in FIG. 10A, the first embodiment of attachment member 12 includes a band 12a along an inside surface near the uppermost edge thereof. Band 12a may be composed of a material having a coefficient of friction greater than that of the material forming the remainder of attachment member 12 for secure gripping of capsule 4 when held in attachment member 12, until it is dislodged therefrom by one of the means described herein. For example, attachment member 12 may be composed of a smooth plastic, and band 12a may be composed of a high-friction rubber. Band 12a may be in the form of an O-ring and may be seated within a groove formed within the inside surface of attachment member 12, as shown in the cross sectional view of FIGS. 9A and 9B. Thus, capsule 4 is securely gripped by band 12a when it is held within attachment member 12.

As shown in FIG. 10B, the second embodiment of attachment member 12 includes a flexible edge 12b at the uppermost edge thereof. Flexible edge 12b may be composed of a material having a coefficient of friction greater than that of the material forming the remainder of attachment member 12 and acts in a manner similar to that of band 12a in FIG. 10A, namely to securely grip capsule 4 within attachment member 12, until it is dislodged therefrom by one of the means described herein. In the embodiment of FIG. 10B, however, the entire uppermost edge 12b of attachment member 12, not just a narrow circumferential band as in FIG. 10A, is formed of this high friction material.

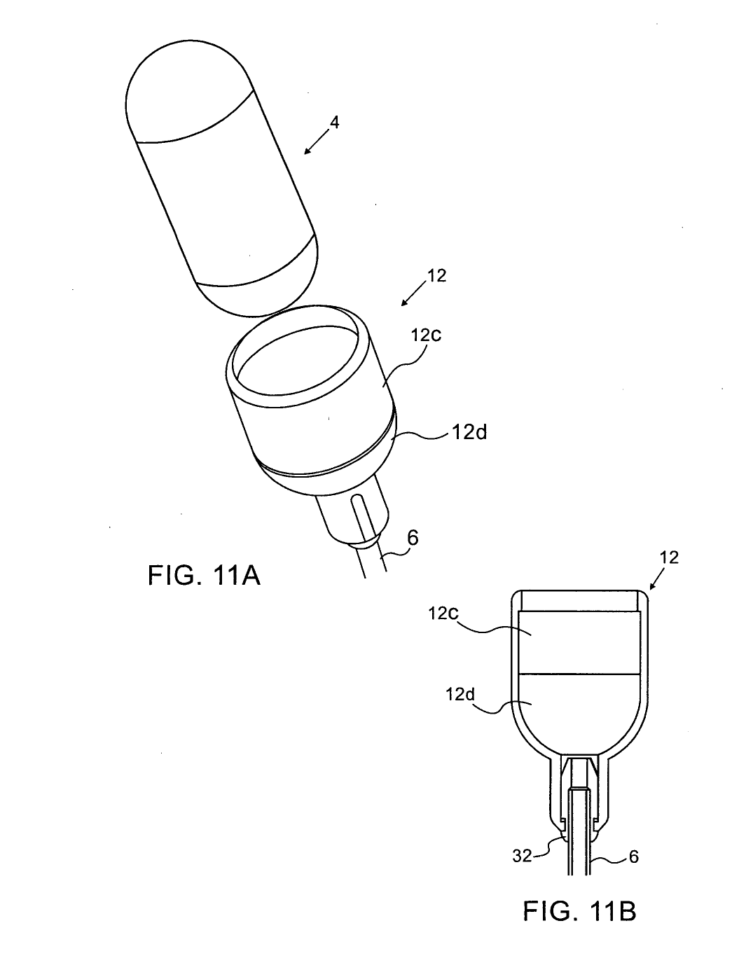

Reference is made to FIGS. 11A and 11B, which show a perspective view and a cross-sectional view, respectively, of a third embodiment of attachment member 12. The third embodiment of attachment member 12 includes a flexible portion 12c and a rigid portion 12d. Similarly to flexible edge 12b of FIG. 10B, flexible portion 12c of FIGS. 11A and 11B may be composed of a material having a coefficient of friction greater than that of the material forming rigid portion 12d and acts to securely grip capsule 4 within attachment member 12, until it is dislodged therefrom by one of the means described herein. In the embodiment of FIGS. 11A and 11B, however, the entire leading portion of attachment member 12, not just a narrow circumferential band as in FIG. 10A or an uppermost edge 12b as in FIG. 10B, is formed of this high friction material. In contrast, rigid portion 12d does not flex, or flexes minimally, in response to forces typically encountered during capsule 4 delivery. Rigid portion 12d rigidly holds capsule 4 in a direction approximately along longitudinal axis 20 of guide apparatus 2 for providing control to direct and manipulate the angle/direction of view of capsule 4 as described hereinabove. Furthermore, rigid portion 12d may provide structural durability near vulnerable joints, for example, where attachment member 12 engages mating element 24.

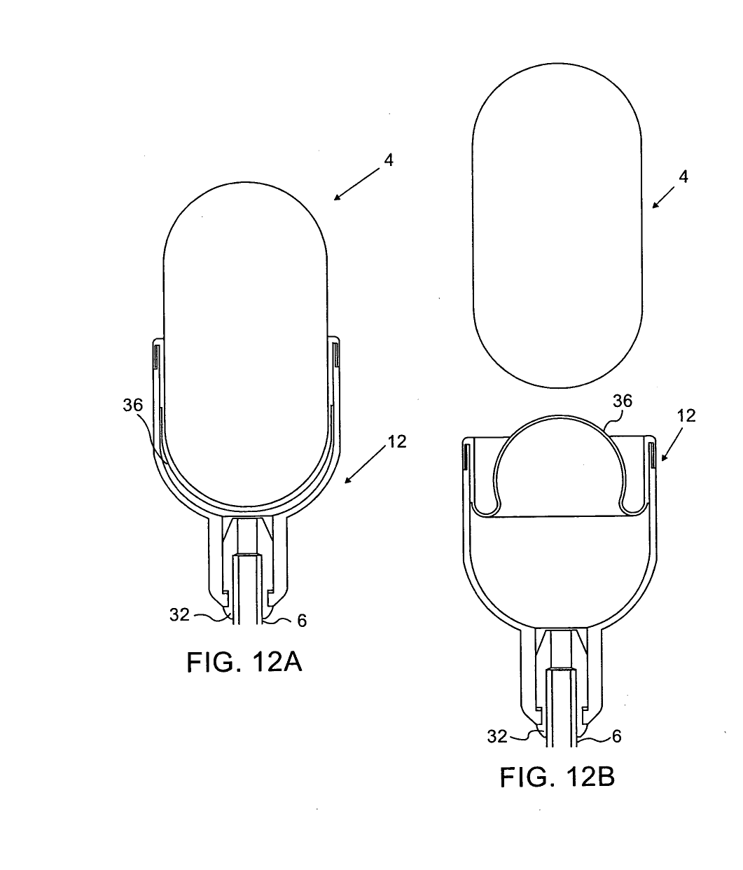

Reference is made to FIGS. 12A and 12B, showing cross-sectional views of a fourth embodiment of attachment member 12. In this embodiment, attachment member 12 may include an invertible member 36, as shown in the perspective views of FIGS. 13A and 13B. Invertible member 36 may be composed of a material having a coefficient of friction greater than that of the material forming attachment member 12 and having sufficient flexibility to deform elastically when inverted without losing its shape and without dislodging from attachment member 12, such as silicon. Invertible member 36 is fitted or glued within the cavity of attachment member 12 and acts to securely grip capsule 4 therein, until it is inverted, whereupon capsule 4 is dislodged therefrom.

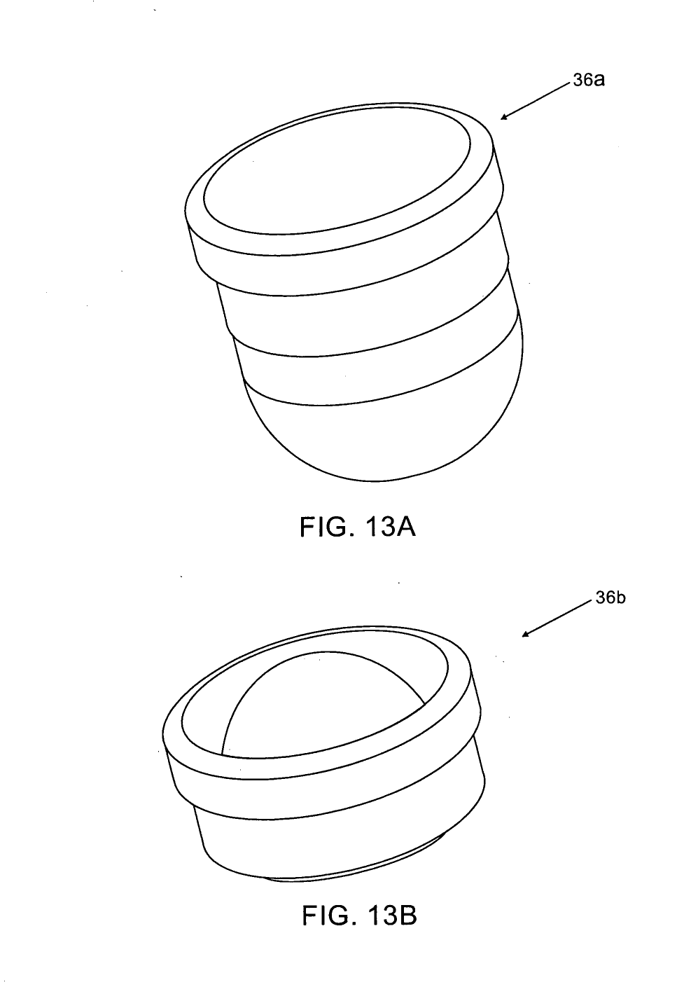

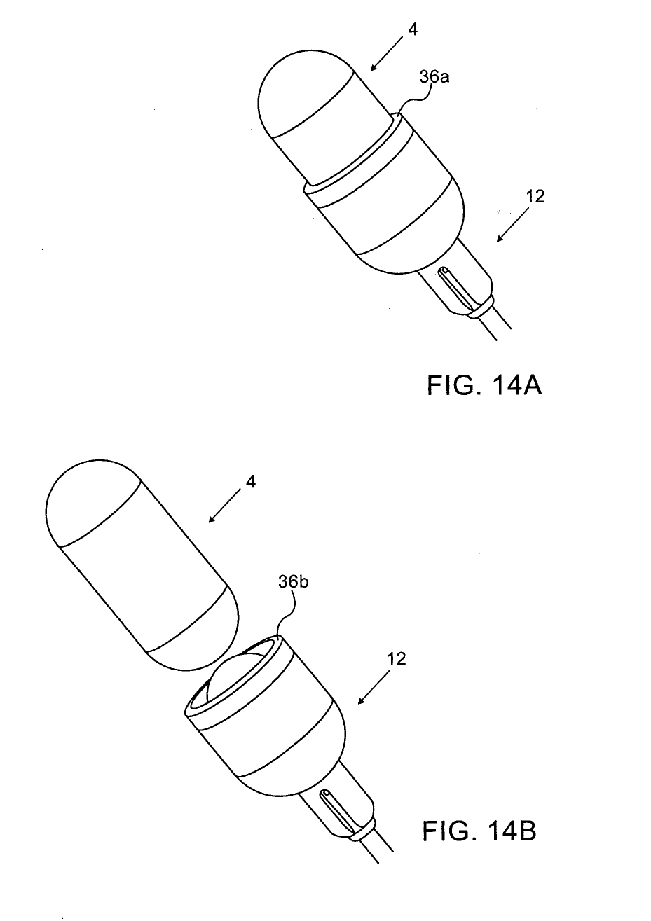

When invertible member 36 is in a concave state 36a, as shown in FIGS. 12A and 13A, capsule 4 is adapted to fit within invertible member 36 and may be securely held by frictional forces. However, invertible member 36 may be inverted via hydraulic or pneumatic pressure from actuator 34. For example, the hydraulic or pneumatic pressure from actuator 34 may inflate invertible member 36 to balloon into convex state 36b, as shown in FIGS. 12B and 13B. When invertible member 36 is in convex state 36b, capsule 4 no longer fits within invertible member 36 and is forced out of attachment member 12. FIGS. 14A and 14B show outside perspective views of attachment member 12 with invertible member 36 disposed within the cavity thereof with capsule 4 disposed therein when invertible member 36 is in concave state 36a and with capsule 4 ejected therefrom when invertible member 36 is in convex state 36b, respectively.

As discussed above, invertible member 36 may be inverted via hydraulic or pneumatic pressure from actuator 34. One advantage of using hydraulic or pneumatic release means with invertible member 36 as opposed to with the high-friction circumferential band of FIG. 10A, uppermost edge 12b of FIG. 10B, and portion 12c of FIGS. 11A and 11B is that capsule 4 is released more smoothly and less abruptly from attachment member 12.

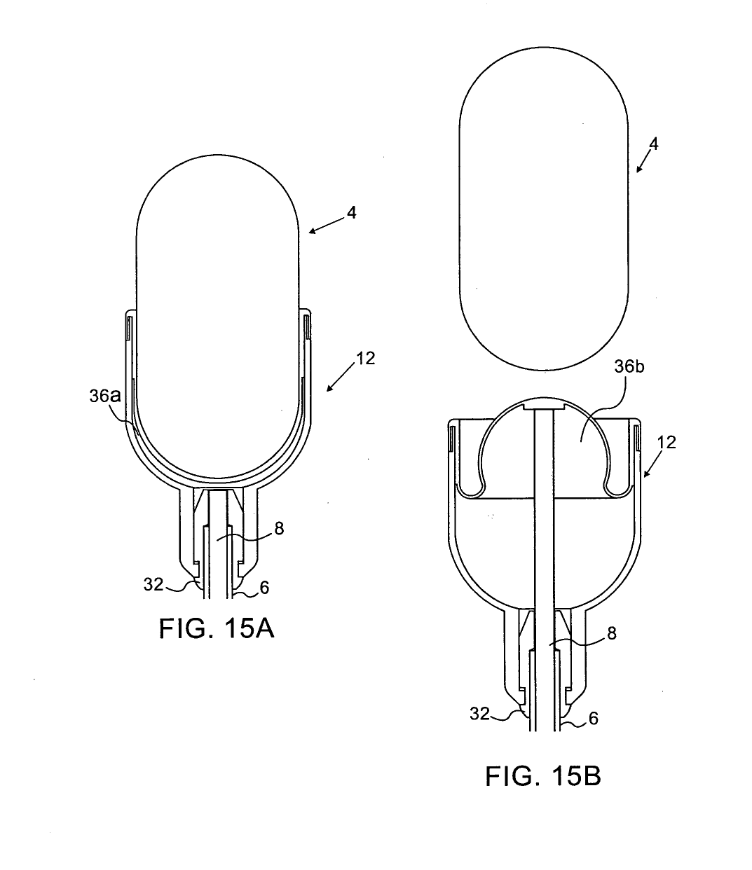

In an alternative embodiment, invertible member 36 may be inverted via mechanical means. FIGS. 15A and 15B show cross-sectional views of attachment member 12 having invertible member 36 being inverted by mechanical means, e.g., physical pressure from shaft 8.

As described hereinabove, shaft 8 traverses hollow sleeve 6 of guide apparatus 2. At the distal tip of sleeve 6, there is an opening 32. When shaft 8 extends beyond opening 32, shaft 8 abuts invertible member 36 to force invertible member 36 from concave state 36a, in which capsule 4 is securely held, to convex state 36bB, in which capsule 4 is released. In addition, in contrast to the embodiment of mechanical release of capsule 4 from attachment means 12 shown in FIGS. 7A and 7B, the inclusion of invertible member 36 between the distal end of shaft 8 and capsule 4 will soften the impact of shaft 8 against capsule 4 and make it is less likely for shaft 8 to cause trauma to capsule 4. This is a particular advantage when the imaging capsule 4 is a “double-headed” capsule, that is, having imaging components and an optical window at both its longitudinal ends, as the optical window may be relatively vulnerable, e.g., as compared to a protective housing, and invertible member 36 ensures that no damage is done to the optical window by ensuring soft impact of shaft 8 against imaging capsule 4.

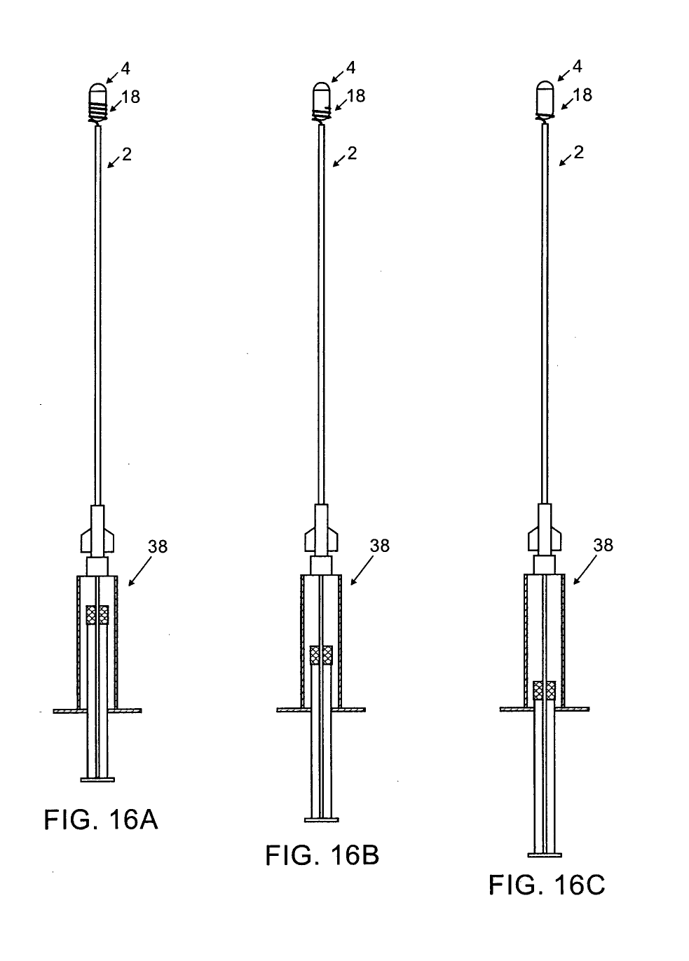

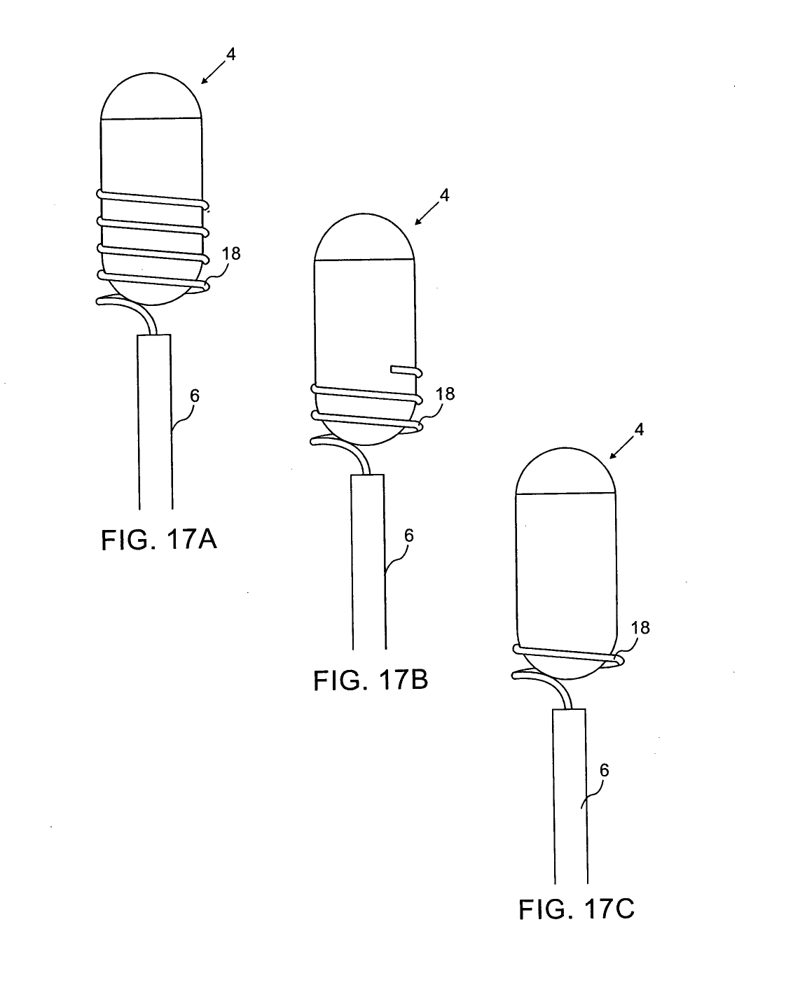

Reference is made to FIGS. 16A, 16B and 16C, showing views of guide apparatus 2 having a third embodiment of a mechanism for holding capsule 4 within and releasing capsule 4 from guide apparatus 2. In this embodiment, guide apparatus 2 includes a retractable coil 18 to grip capsule 4 and to release capsule 4. FIGS. 17A, 17B and 17C show closer views of FIGS. 16A, 16B and 16C, respectively, showing attachment member 12 having a retractable coil 18 at the distal end of shaft 8 extending out of the distal end of guide apparatus 2.

In operation, retractable coil 18, which is the distal end of shaft 8, is initially held within sleeve 6. After shaft 8 is passed through endoscope 26, as shown in FIG. 6, shaft 8 is pushed distally, such that the distal end of shaft 8 protrudes from sleeve 6 to form retractable coil 18. The user then threads capsule 4 into retractable coil 18 by applying force. Capsule 4 is thus tightly fitted within the winding of the retractable coil 18. It is preferred that the distal tip of retractable coil 18 have a rounded end so as not to damage capsule 4 during this attachment process.

As shown in FIGS. 16A-17C, the distal end portion 18 of shaft 8 is preformed with coiled shape. When distal end portion 18 of shaft 8 extends through the distal end of hollow sleeve 6 of guide apparatus 2, this portion takes its naturally coiled shape as a retractable coil 18. At its proximal end, shaft 8 is mechanically connected to an actuator 38 to controllably retract shaft 8 into sleeve 6 and to control the length of the retractable coil 18 protruding from the distal opening of sleeve 6. Alternatively, a controller 30, as shown in FIG. 6, may be used to controllably retract shaft 8 toward the proximal end of sleeve 6. The more that actuator 38 or controller 30 proximally retracts shaft 8, the less the length of retractable coil 18 protrudes beyond the distal opening of sleeve 6.

The release of capsule 4 is done by retracting shaft 8 such that retractable coil 18 is pulled into sleeve 6. When actuator 38, or controller 30, completely (or nearly completely) retracts retractable coil 18, the length of retractable coil 18 protruding outside sleeve 6 is insufficient to hold capsule 4. Capsule 4 is thereby released.

In one embodiment, there may be a safety mechanism (not shown) built into the distal end of guide apparatus 2, e.g., acting as a gate to opening 32, beyond which shaft 8 cannot extend. A controller for the safety mechanism (not shown) may be located at the proximal end of guide apparatus 2 for ease of access by an administrator while guide apparatus 2 is in use. The safety mechanism may be controlled by an actuating means, e.g., a latch or button. When the control is actuated, the safety mechanism is dismantled to allow shaft 8 to extend into opening 32 to force a mounted capsule 4 to release. Alternatively, a safety mechanism may be built into or attached to guide apparatus 2 at its proximal end, e.g., at hydraulic actuator 34 of FIGS. 8A and 8B.

Other means for securing and releasing capsule 4 may be used according to some examples as follows. In one alternative embodiment, shaft 8 may dislodge capsule 4 by a mechanical force, as described above. However, in this embodiment, a threaded tip of shaft 8 may be used to screw through a threaded opening 32 by manipulation of controller 30 by a screwing action at the proximal end of guide apparatus 2. In another alternative embodiment, capsule 4 is held by a suction (vacuum) force. A suction device may be positioned to provide suction pressure through the proximal end of sleeve 6 to hold capsule 4 at the distal end of sleeve 6. When the suction pressure is turned off (or reversed), capsule 4 is released from guide apparatus 2. In another alternative embodiment, attachment member 12 is composed of a highly flexible and foldable material, e.g., rubber, tethered via a cord extending through sleeve 6 (in parallel with shaft 8) to the proximal end of guide apparatus 2. To release capsule 4, the tether is pulled through sleeve 6. Attachment member 12 folds and is retracted proximally into the opening of sleeve 6, while capsule 4 is pinched off by the edge of the distal tip of sleeve 6 and released from guide apparatus 2. In another alternative embodiment, capsule 4 is held by a magnetic force. Attachment member 12 and the capsule 4 may have magnets of opposite polarity. Guide apparatus 2 may have a switch at the proximal end (outside the patient) for turning off the magnet or switching the polarity of the magnet of the attachment element to repel capsule 4. Other mechanisms for holding and releasing capsule 4 may be used.

After the procedure is finished and capsule 4 is delivered and dispensed into the stomach of a patient, endoscope 26 and guide apparatus 2 are pulled out through the esophagus and removed from the patient. In one embodiment, attachment member 12 and mating element 24 are unlocked. Alternatively, if there is no other means to remove the attachment member 12, in order to remove guide apparatus 2 from endoscope 26, shaft 8 is retracted, and guide apparatus 2 is cut, ripped or broken along sleeve 6 (to break off attachment member 12). Guide apparatus 2 is pulled back through endoscope 26 and then discarded.

Sleeve 6 may be composed of any elastic material having a modulus of elasticity sufficient to return to its original shape after being deformed. For example, such materials may include polymers, rubber, etc.

Shaft 8 may be composed of a material having sufficient rigidity and stiffness to be able to straighten bend 14 of sleeve 6. For example, such materials may include wire made of a metal such as steel, a shape memory alloy such as Nitinol, etc., or any other material having sufficient stiffness and rigidity but having a memory for a preformed shape The material may be covered or glazed with a low-friction polymer material to increase the smoothness of shaft 8 and to decrease its surface friction.

The portion of attachment member 12 for holding capsule 4 may be composed of a biocompatible polymer, e.g., polycarbonate, acetal, rubber, etc. This portion may be mostly rigid, but typically can bend slightly when external forces are applied thereto.

The portion of attachment member 12 for locking to mating element 24 is rigid. This portion may be composed of metal such as aluminum or hard plastics.

It may be appreciated by those skilled in the art that shaft 8 need not be perfectly straight. For example, shaft 8 may be slightly bent with respect to longitudinal axis 20 or, in another embodiment, shaft 8 may be a coil spiraling about longitudinal axis 20.

It may be appreciated by those skilled in the art that although guide apparatus 2 is shown to have a single bend 14 having a specific curvature, multiple bends may be used along the length of sleeve 6, which may be of any and optionally different curvatures. In one embodiment, sleeve 6 may have the shape of one long bend extending its whole length. In this example, sleeve 6 may be packaged as a wound coil.

Although aforementioned embodiments of guide apparatus 2 describe shaft 8 as a straight and highly rigid body traversing a bent and flexible sleeve 6, in an alternate embodiment, both shaft 8 and sleeve 6 are flexible and, instead, endoscope 26 is the rigid body used to straighten guide apparatus 2. In particular, while the portion of guide apparatus 2 fully enclosed by endoscope 26 conforms to its straight shape, the portion of guide apparatus 2 protruding outside endoscope 26 experiences no restraining force and, to the extent not also restrained by the body lumen, returns to its natural bent shape. In this embodiment, guide apparatus 2 is straightened by proximally retracted shaft 8 and sleeve 6 into endoscope 26 and bent by pushed shaft 8 and sleeve 6 distally out of endoscope 26 so that there is no substantial external force thereon. Such a guide apparatus 2 may be adapted to move in all directions discussed above in reference to FIG. 4 for capsule 4 to view 360° in all directions.

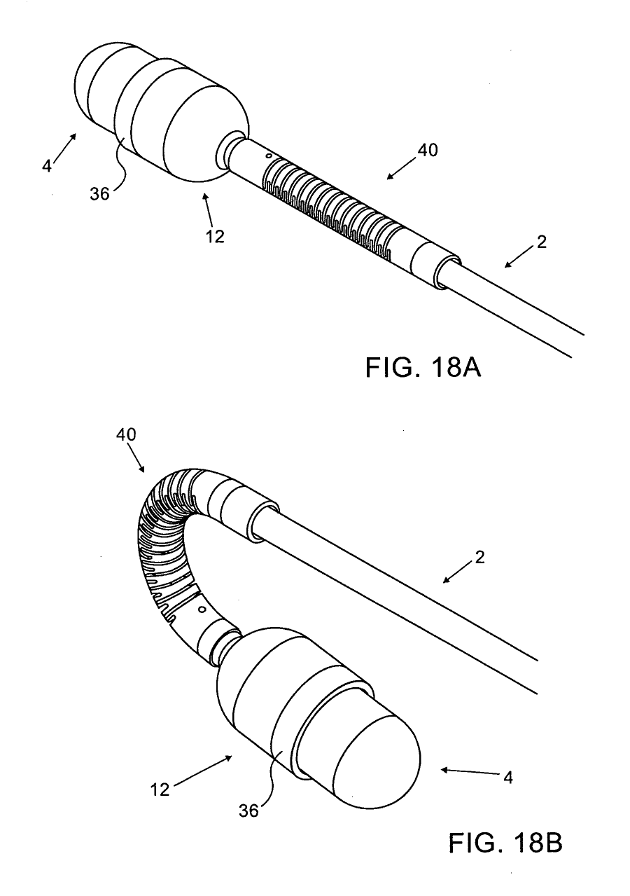

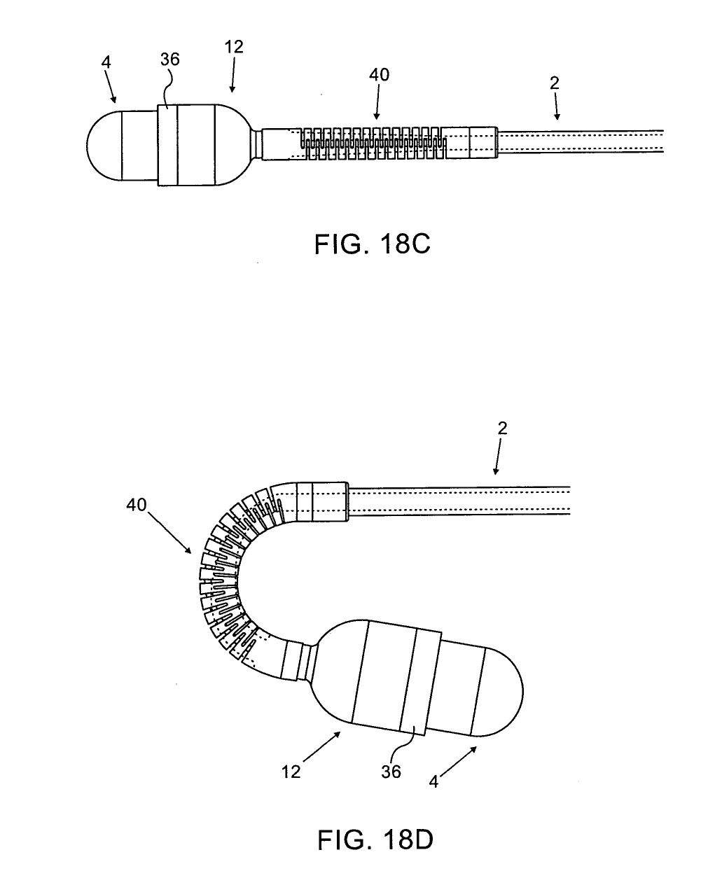

FIGS. 18A-23C depict a guide apparatus that is a stand-alone device. This device may overcome the need of passing a guide 2 holding the capsule through a working channel of an endoscope. Instead of passing a guide through an endoscope, capsule 4 may be attached to a guide apparatus 2, which may have an integral bending section 40, as will be described below. The capsule 4 may be attached to the integral bending section 40 through attachment member 12 with invertible member 36 disposed within the cavity thereof and with capsule 4 disposed therein. During insertion of the integral bending section 40 into the patient's stomach, the patient may begin to swallow the capsule 4 (which is already disposed within invertible member 36 attached to the guide 2). Right after swallowing the capsule 4, or during that step, the operator, e.g., a physician, may gently push the integral bending section 40 further into the patient's esophagus and then into the patient's stomach. In some embodiments, bending section 40 may comprise means for enabling insufflation of the stomach so as to provide a better view of the stomach walls.

Reference is now made to FIGS. 18A and 18B which show a back and side perspective view and a front and side perspective view, respectively, of a guide apparatus 2 and attached capsule 4 in accordance with another embodiment of the invention. In some embodiments, bending section 40 may be made of Nitinol. Bending section 40 may be made of a Nitinol tube which may include laser cuts about the circumference thereof, so as to enable the Nitinol tube 40 to bend. The laser cuts in the Nitinol tube may provide the Nitinol, which is rigid when is in a straight configuration, with flexibility. The design of the cuts in the Nitinol tube may determine the range of the bending angle. In some embodiments, the Nitinol tube 40 may be designed to bend up to 180 degrees. In practice, the bending angle may be less than 180 degrees, since, as described in FIG. 4, the capsule 4 has its own angle/direction of view which may be added to the bending angle in order to achieve a angle/direction of view in an angle of 180 degrees on both sides of the bending section 40.

Reference is now made to FIGS. 18C and 18D which show side perspective views of the guide apparatus 2 and attached capsule 4 of FIGS. 18A and 18B, respectively. In some embodiments, integral bending section 40 may have two pull-wires 42 passed through it and attached to its proximal end, i.e., near the end where attachment member 12 is attached to bending section 40. Pull-wires 42 may be used to bend the bending section, e.g., Nitinol tube 40. Nitinol tube 40 may bend to either side according to which pull-wire 42 is pulled. In some embodiments, the amount of tension of the pull-wire 42 controls the size of the bending angle of Nitinol tube 40. The more either pull-wire 42 is pulled, the larger the bending angle is in the direction of that pull-wire.

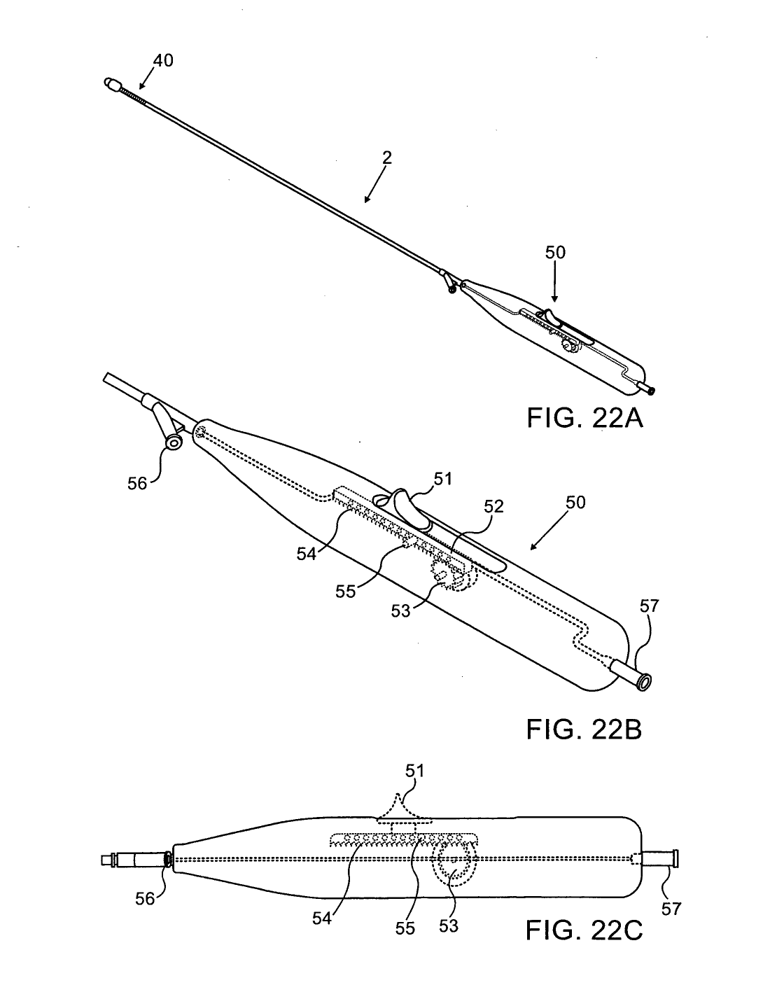

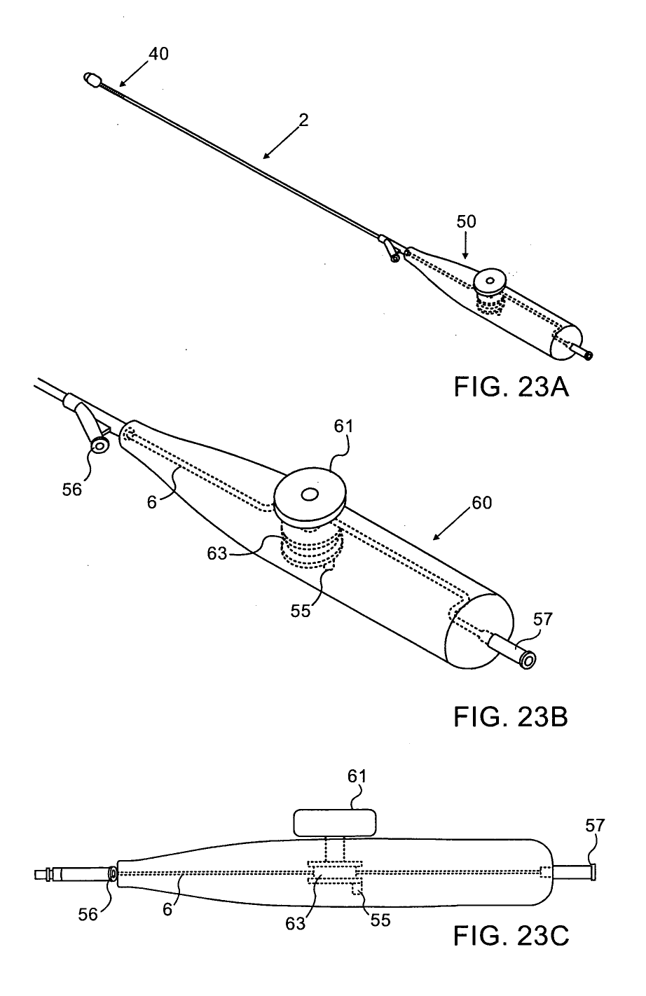

According to some embodiments, when the guide is inserted into the stomach there is a need for insufflation in addition to the need for bending capabilities of the guide apparatus. In embodiments in which the stomach must be collapsed in order to achieve a good view of its walls, there is a need to insufflate the stomach. In some embodiments, air may be supplied into the guide apparatus and then to the integral bending section 40 through an opening in the main tube 2, as will be described later with regard to FIGS. 22-23. In order to allow passage of air through the bending section 40 and into the stomach, bending section 40 may comprise holes. In this embodiment, Nitinol tube 40 is laser cut so as to acquire flexibility. The cuts 41 in the Nitinol tube 40 may provide flexibility but may also provide holes through which air may enter into the stomach and cause it to inflate.

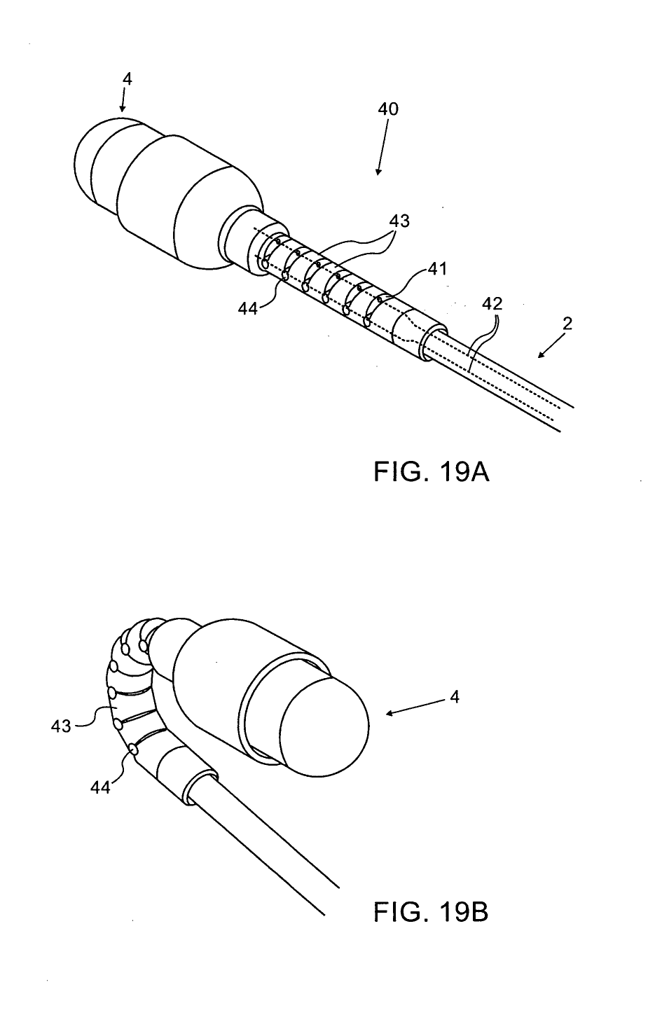

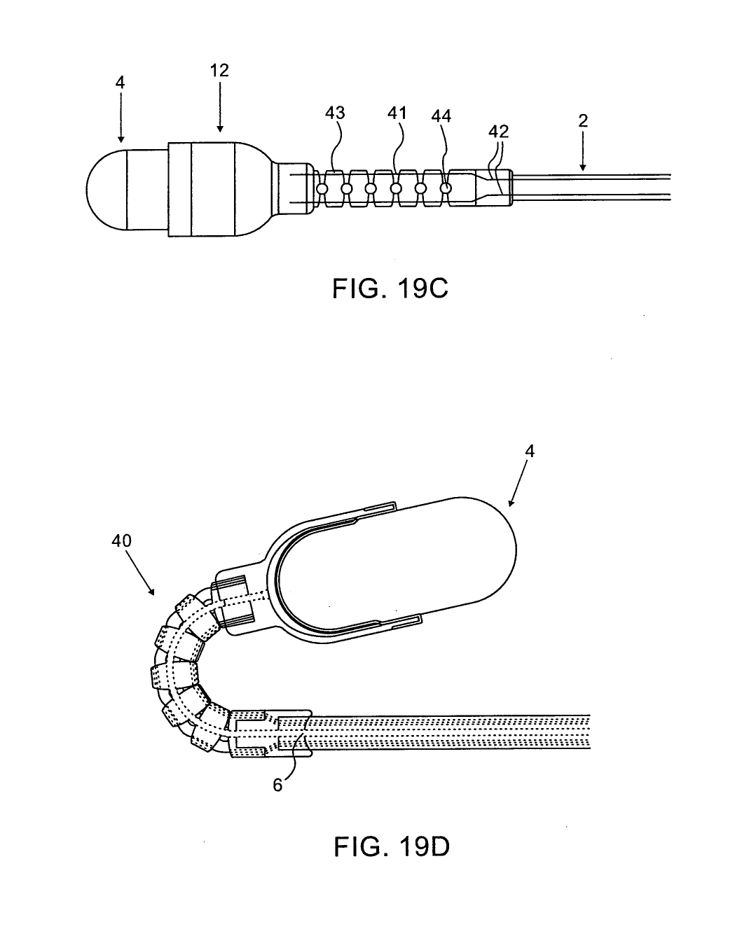

Reference is now made to FIGS. 19A and 19B which show back and side and front and side perspective views of a guide apparatus and attached capsule in accordance with yet another embodiment of the invention. FIGS. 19A-19B show an integral bending section 40 of a different kind than that shown in FIGS. 18A-D. According to this embodiment, bending section 40 may comprise individual sections 43 made from, e.g., plastic parts, which may be connected to one another through hinges 44 and may bend around hinges 44. Individual sections 43 connected through hinges 44 may create a “caterpillar like” tube. When one of pull-wires 42 is pulled by the operator of the guide apparatus, the individual sections 43 may come close to one another around hinges 44 from one of their sides, until they touch each other on that side (shown in FIG. 19B).

This configuration of bending section 40 comprising individual sections 43 provides flexibility, but, in order to provide rigidity specifically during insertion of the guide apparatus through the patient's mouth, pull-wires 42 should both be kept at a certain tension. After insertion into the patient's stomach and while pulling one of the pull-wires 42, in order to bend the bending section 40 so as to acquire images of all sides of the stomach walls, the other pull-wire 42 should also be held at a certain tension so that the tube may acquire intermediate bending angles. When the individual sections 43 touch each other, they create the maximum bending angle possible. When in the maximum bending angle, the contact between the individual sections 43 provides rigidity to the bending section 40. However, in order to provide rigidity in intermediate angles, the other pull-wire 42 (which is not the one pulled for bending the plastic parts 43) should also be pulled at a certain tension so as not to have too much slack and be loose.

FIGS. 19C-19D show a side and cross section of the bending section described in FIGS. 19A-19B. As discussed above, the bending section 40 should comprise holes for air passage in order to have the ability to insufflate the stomach when desirable. According to this embodiment, the individual sections 43 may be hollow and may be in a shape which creates grooves 41. Since individual sections 43 are connected through hinges 44 and should have the ability to bend to either side around the hinges 44, the individual sections 43 may comprise grooves 41 on opposite sides along the longitudinal axis of the bending section 40. Grooves 41 may provide the space needed for individual sections 43 to bend over and also may provide holes through which air may exit the bending section 40 and enter the stomach.

FIG. 19D shows a cross-section of the bending section 40 and the attachment member 12 which holds capsule 4. In some embodiments, a hollow sleeve 6 may be passed through main tube 2 and then through bending section 40 into attachment member 12 which includes invertible member 36. Invertible member 36 may be inverted via hydraulic or pneumatic pressure from actuator 34 which passes through hollow sleeve 6 into invertible member 36. Gas (e.g., air or oxygen) or fluid (e.g., water or saline) may be pressurized through hollow sleeve 6 and into invertible member 36 so as to invert invertible member 36 and dislodge capsule 4 from guide apparatus 2.

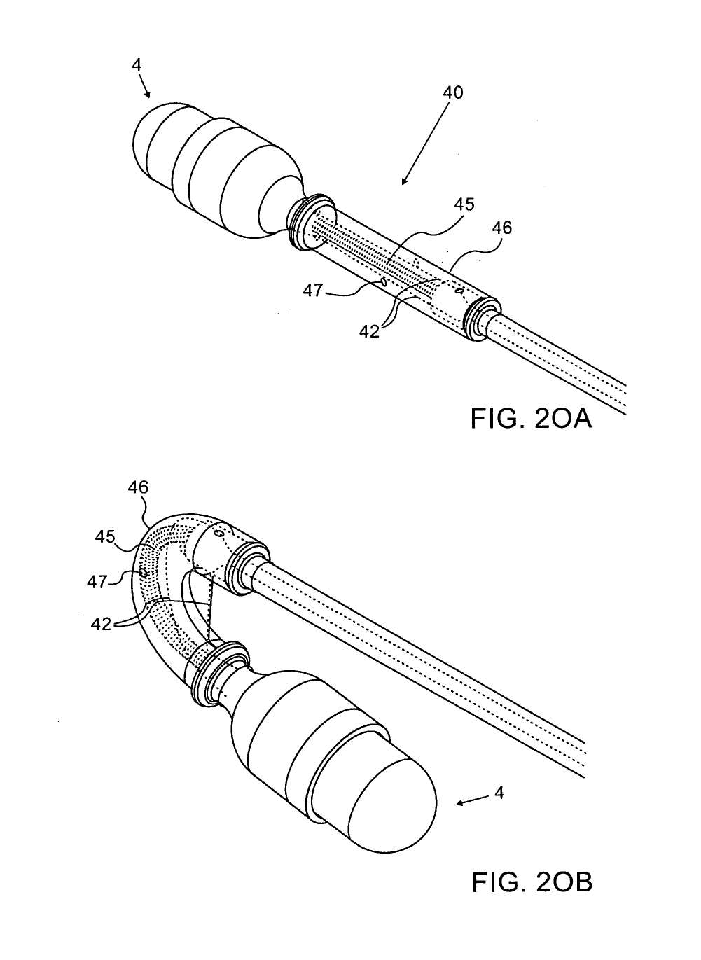

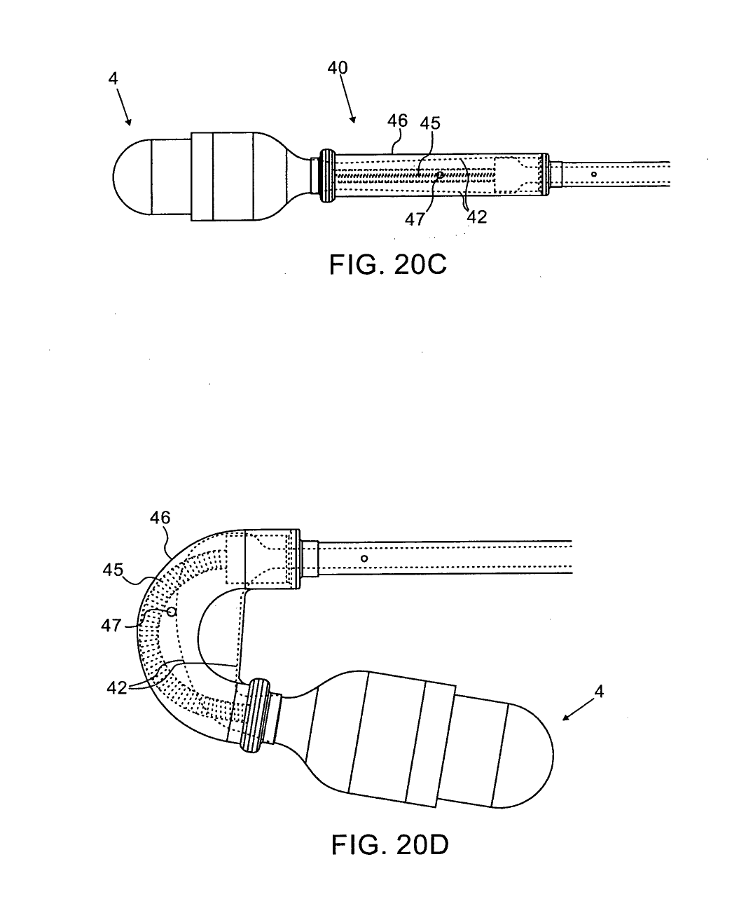

FIGS. 20A-20D show back-side and front-side and side perspective views of a guide apparatus and attached capsule in accordance with a third embodiment of the invention. In this embodiment, bending section 40 may comprise a spring 45 which may be covered by a hollow cover 46. Cover 46 is typically made of a flexible and elastic material, e.g., silicon. Cover 46 may comprise holes 47 through which air may exit and thus enter the stomach to insufflate it. Cover 46 may prevent tissue from getting caught within the coils of spring 45 when the spring 45 is in an angled configuration or in a straight configuration. Bending section 40, according to this third embodiment, may comprise two pull-wires 42 which may be positioned on opposite sides of the spring 45. The pull-wires 42 may be passed through guide apparatus 2, and their distal ends may be securely attached within bending section 40. When one of pull-wires 42 is pulled, the spring 45 may bend so as to provide a wide angle/direction of view. In order to bend the spring in a substantially 180 degrees bending angle, one of the pull-wires 42 needs to actually be outside cover 46 (FIGS. 20B, 20D). Since a smaller bending angle may be used, when taking into consideration the angle/direction of view of the capsule 4, then this problem may be overcome.

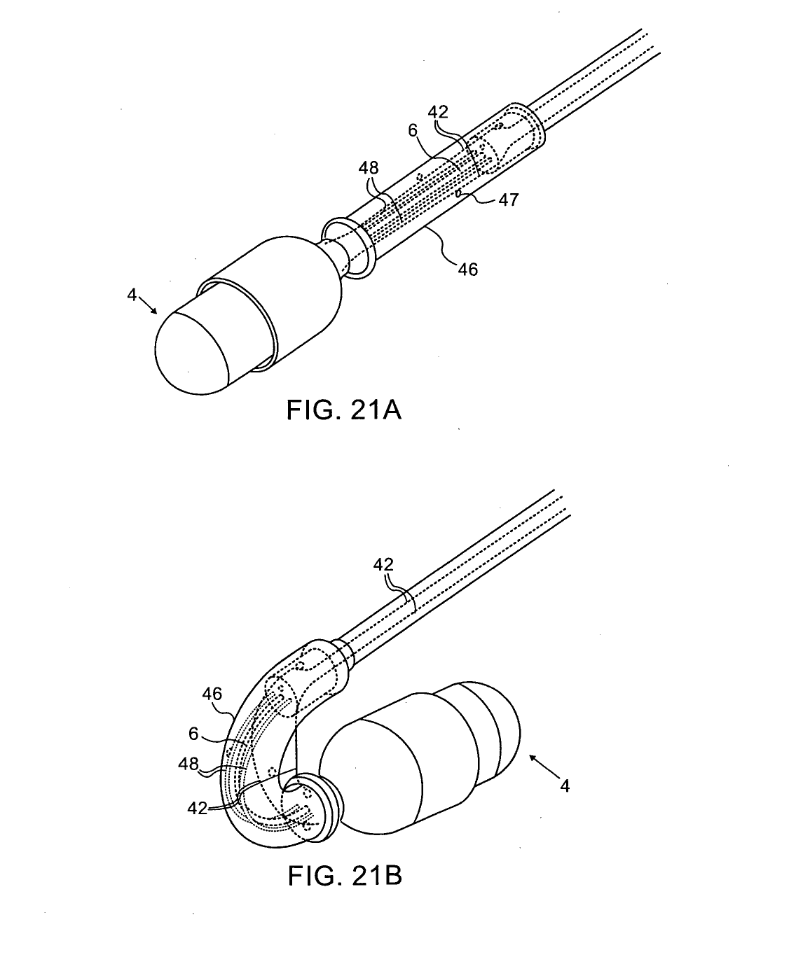

Reference is now made to FIGS. 21A-21B which show a front and side and back and side perspective views of a guide apparatus and attached capsule in accordance with a fourth embodiment of the invention. In this embodiment, the integral bending section 40 comprises two Nitinol wires 48 which may be covered with hollow cover 46. Cover 46 is typically made of a flexible and elastic material, e.g., silicon. Cover 46 may comprise holes 47 through which air may exit and thus enter the stomach to insufflate it. Cover 46 may prevent tissue from getting caught between the Nitinol wires 48 or between the pull-wires 42 and the Nitinol wires 48. Bending section 40, according to this fourth embodiment, may comprise two pull-wires 42 which may be positioned on the outer sides of the Nitinol wires; each pull-wire 42 may be positioned such that a Nitinol wire 48 is on one of its sides and on the other side is the inner wall of cover 46. The pull-wires may be passed along guide apparatus 2 and be securely attached within bending section 40. When one of pull-wires 42 is pulled, the Nitinol wires 48 may bend so as to provide a wide angle/direction of view. In order to bend the Nitinol wires 48 in a substantially 180 degrees bending angle, one of the pull-wires 42 needs to actually be outside cover 46 (FIG. 21B). Since a smaller bending angle may be used, when taking into consideration the field of view of the capsule 4, then this problem may be overcome.

Reference is now made to FIGS. 22A-22C and FIGS. 23A-23B which show a mechanism for controlling orientation of the capsule according to two embodiments of the present invention. In FIGS. 22A-22C, the mechanism for controlling the two-pull-wires 42 may comprise a sliding knob 51. Sliding knob 51 may be moved backwards and forwards along track 52. Sliding knob 51 may be attached to a flat bar with teeth 54 which may intermesh with a gear pulley 53, like a rack and pinion which may control the pull-wires 42 tension. When sliding knob 51 is moved by the operator, the rack 54 interlocks with the pinion, i.e., the gear pulley 53. This sliding mechanism may comprise a position lock plunger spring 55 which may assist in maintaining a certain position of the sliding knob 51 and such maintain a certain tension of the pull-wires 42 which eventually correlates to the size of bending angle. Subsequent to sliding the knob 51, the position lock plunger spring 55 snaps in between the teeth of rack 54 so as to lock the sliding knob 51 from further sliding. The spring power of position lock plunger spring 55 is easy to overcome when the operator applies some force if it is desired to change the bending angle. However, the position lock plunger spring 55 may provide some stability when the operator stops the sliding motion, in keeping the bending angle constant by keeping constant tension in the pull-wires 42. This may enable the operator to perform other procedures while the bending angle is kept constant (e.g. the operator may turn the entire device around its longitudinal axis to get a 360 degrees angle/direction of view).

In some embodiments, the controlling mechanism may comprise an opening 56, to which an air supply may be connected. Typically opening 56 may comprise a Luer connector, which are common connectors used in the medical field. Many devices contain Luer locks and Luer connectors, so this may comply with standard equipment present in hospitals and clinics. In other embodiments, other connectors may be used.

In some embodiments, the controlling mechanism may comprise a connector 57 for attaching the hydraulic/pneumatic mechanism, e.g. syringe, to the guide apparatus. Connector 57 may be connected to hollow sleeve 6, through which gas or fluid may pass in order to insufflate the invertible member 36 which thereby releases the capsule 4 out of its hold. Typically connector 57 is a Luer connector.

In FIGS. 23A-23C, the mechanism 60 for controlling the two pull-wires 42 may comprise a rotating knob 61. Rotating knob 61 may be attached to a pulley 63 around which the pull-wires 42 may be coiled. This rotating mechanism 60 may further comprise a position lock plunger spring 55 which may assist in maintaining a certain position of the rotating knob 61 and, as such, maintain a certain tension of the pull-wires 42, which ultimately correlates to the size of bending angle. Subsequent to rotation of knob 61, the position lock plunger spring 55 snaps against pulley 63 so as to lock the rotating knob 61 from further rotating. The spring power of position lock plunger spring 55 is easy to overcome when the operator applies some force while beginning to rotate the rotating knob 61 again, if it is desired to change the bending angle. However the position lock plunger spring 55 may provide some stability when the operator stops the rotating motion, in keeping the bending angle constant by keeping constant tension in the pull-wires 42. This may enable the operator to perform other procedures while the bending angle is kept constant (e.g., the operator may turn the entire device around its longitudinal axis to get a 360 degrees angle/direction of view).

In some embodiments, the controlling mechanism may comprise an opening 56, to which air supply may be connected. Typically opening 56 may comprise a Luer connector or any other connector.

In some embodiments, the controlling mechanism may comprise a connector 57 for attaching the hydraulic/ pneumatic mechanism, e.g., syringe to the guide apparatus. Connector 57 may be connected to hollow sleeve 6 through which gas or fluid may pass in order to insufflate the invertible member 36 which thereby releases the capsule 4 out of its hold. Typically connector 57 is a Luer connector.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the scope of the invention.

What is claimed is:

1. A guide for an endoscope capsule, the guide comprising:
a hollow sleeve having a proximal end and a distal end;
an attachment element for mounting the capsule, said attachment element attached to the distal end of said sleeve, said attachment element having a cavity; and
an invertible member for fitting said capsule within, said invertible member positioned within said cavity and attached to said attachment element, wherein said invertible member is inverted via hydraulic or pneumatic pressure to expel said capsule from the attachment element.

2. The guide of claim 1, wherein the guide further comprises an actuator, said actuator comprising a cavity containing a fluid and an actuating member to pressurize the fluid in the cavity, thereby inverting the invertible member.

3. The guide of claim 2, wherein said fluid is selected from a group consisting of: water, saline solution and air.

4. The guide of claim 1, wherein said sleeve comprises a mating element for securing said mounting element onto said sleeve.

5. The guide of claim 4, wherein the mating element is attached to the sleeve by attachment means selected from a group consisting of: a luer lock, a clip, a snap, a detent mechanism, a screw and a magnet.

6. The guide of claim 1, wherein the guide is contained within an endoscope.
